(19) United States
(12) Patent Application Publication
(10) Pub. No.: US 2020/0405997 A1
(43) Pub. Date: Dec. 31, 2020

(54) SYSTEMS FOR EMITTING LIGHT WITH TUNABLE CIRCADIAN EFFECTS AND SUBSTANTIALLY CONSISTENT COLOR CHARACTERISTICS AND METHODS OF MAKING AND/OR OPERATING THE SAME

(71) Applicants: Qifeng Shan, San Jose, CA (US); Hien Lam, Tracy, CA (US); Xufang Chen, Fremont, CA (US); Thomas R. Jory, Santa Cruz, CA (US)

(72) Inventors: Qifeng Shan, San Jose, CA (US); Hien Lam, Tracy, CA (US); Xufang Chen, Fremont, CA (US); Thomas R. Jory, Santa Cruz, CA (US)

(21) Appl. No.: 16/452,733

(22) Filed: Jun. 26, 2019

(51) Int. Cl. A61M 21/00; H05B 33/08; H05B 37/02; H01L 33/50

(52) U.S. Cl. CPC A61M 21/00; H05B 33/086; A61M 2021/0044; H01L 33/504; H05B 37/0281

(57) ABSTRACT

Aspects of the present disclosure relate to systems for emitting light (e.g., substantially white light) with tunable circadian effects and substantially consistent color characteristics, and methods of making and/or operating the same. Certain embodiments described herein are systems comprising a plurality of light-emitting regions configured to emit light having certain circadian effects (e.g., melanopic ratio) and certain color characteristics (e.g., corrected color temperature (CCT), color rendering index (CRI)). According to some embodiments, the difference between the circadian effects of the light-emitting regions may be relatively large, and the difference between the color characteristics of the light-emitting regions may be relatively small. Each light-emitting region may comprise one or more light-emitting diodes (LEDs), each of which may be associated with one or more wavelength-converting materials (e.g., phosphors).

### FIELD OF THE INVENTION

Systems for emitting light with tunable circadian effects and substantially consistent color characteristics, and methods of making and/or operating the same, are generally described.

### BACKGROUND

In humans and many animals, the circadian rhythm (i.e., sleep-wake cycle) is at least partially regulated by melatonin, which is a hormone produced by the pineal gland in the brain. For example, synthesis and secretion of melatonin may promote sleep onset, while suppression of melatonin may promote behavioral arousal. The secretion or suppression of melatonin may, in turn, be affected by the type of light absorbed by photoreceptors in the eye.

Human eyes contain intrinsically photosensitive retinal ganglion cells (ipRGCs), some of which contain a photopigment called melanopsin. In FIG. 1A, curve 110 represents a plot of an exemplary circadian action function C(λ) (also referred to as a melanopic sensitivity function or a melanopic curve), which indicates the range of wavelengths to which melanopsin-containing ipRGCs are sensitive. The data for exemplary curve 110 can be found in Table L2 of the WELL Building Standard published by the International Well Building Institute (https://standard.wellcertified.com/tables#melanopicRatio). As shown in FIG. 1A, melanopsin-containing ipRGCs are generally sensitive to light having wavelengths of about 400 to about 600 nanometers (nm), with peak absorption around 490 nm. Stimulation of melanopsin-containing ipRGCs (e.g., through absorption of light) may activate the melanopsin signaling system and suppress melatonin synthesis.

In addition to ipRGCs, human eyes also contain rods and cones, which are involved in visual processing. In FIG. 1A, curve 120 represents a plot of visual efficiency function V(λ) (also referred to as a photopic luminosity function or a visual curve), which indicates the range of wavelengths to which certain rods and/or cones are sensitive (i.e., the average sensitivity of human visual perception). The data for exemplary curve 120 can be found in Table L2 of the WELL Building Standard published by the International Well Building Institute (https://standard.wellcertified.com/tables#melanopicRatio). As shown in FIG. 1A, rods and/or cones are generally sensitive to visible light in the range of about 430 nm to about 680 nm, with peak absorption around 555 nm.

It has been recognized that artificial light sources, such as light-emitting diodes (LEDs), can affect not only visual perception, but circadian effects on the human body. As a result, there has been demand for artificial light sources that can emit light having different circadian effects at different times. However, conventional approaches have resulted in light sources for which different levels of circadian effects are associated with different color characteristics. Accordingly, there is a need for improved light sources.

### SUMMARY

The present invention generally relates to systems for emitting light with tunable circadian effects and substantially consistent color characteristics and methods of making and/or operating the same.

Certain aspects relate to a light-emitting system. In some embodiments, the system comprises a first light-emitting region. In certain embodiments, the first light-emitting region is configured to emit light having a first melanopic ratio. In certain embodiments, the first light-emitting region is configured to emit light having a first correlated color temperature (CCT) value. In some embodiments, the system comprises a second light-emitting region. In certain embodiments, the second light-emitting region is configured to emit light having a second melanopic ratio. In certain embodiments, the second light-emitting region is configured to emit light having a second CCT value. In some embodiments, the first melanopic ratio and the second melanopic ratio have a difference of at least 0.1. In some embodiments, the first CCT value and the second CCT value have a difference of 1000 K or less.

According to some embodiments, a light-emitting system is provided. In some embodiments, the system comprises a first light-emitting region. In certain embodiments, the first light-emitting region comprises a first light-emitting diode (LED). In some instances, the first LED is associated with a first wavelength-converting material. In some embodiments, the system comprises a second light-emitting region. In certain embodiments, the second light-emitting region comprises a second LED. In some instances, the second LED is associated with a second wavelength-converting material. In some embodiments, a first combination comprising the first LED and the first wavelength-converting material is configured to emit light having a first melanopic ratio and a first correlated color temperature (CCT) value. In some embodiments, a second combination comprising the second LED and the second wavelength-converting material is configured to emit light having a second melanopic ratio and a second CCT value. In some embodiments, the first melanopic ratio and the second melanopic ratio have a difference of at least 0.1. In some embodiments, the first CCT value and the second CCT value have a difference of 1000 K or less.

Certain aspects relate to a method. In some embodiments, the method comprises emitting light having a first melanopic ratio and a first CCT value. In some embodiments, the method comprises emitting light having a second melanopic ratio and a second CCT value. In some embodiments, the first melanopic ratio and the second melanopic ratio have a difference of at least 0.1. In some embodiments, the first CCT value and the second CCT value have a difference of 1000 K or less.

Other aspects, embodiments, and features of the present invention will become apparent from the following detailed description of various non-limiting embodiments of the invention when considered in conjunction with the accompanying figures. In cases where the present specification and a document incorporated by reference include conflicting and/or inconsistent disclosure, the present specification shall control. If two or more documents incorporated by reference include conflicting and/or inconsistent disclosure with respect to each other, then the document having the later effective date shall control.

### BRIEF DESCRIPTION OF DRAWINGS

Non-limiting embodiments of the present invention will be described by way of example with reference to the accompanying figures, which are schematic and are not intended to be drawn to scale. In the figures, each identical or nearly identical component illustrated is typically represented by a single numeral. For purposes of clarity, not every component is labeled in every figure, nor is every component of each embodiment of the invention shown where illustration is not necessary to allow those of ordinary skill in the art to understand the invention. In the figures:

FIG. 1A illustrates an exemplary circadian action function and an exemplary visual efficiency function;

FIG. 1B illustrates an exemplary CIE 1960 chromaticity plot;

FIG. 2A illustrates, according to some embodiments, an exemplary system comprising a first light-emitting region and a second light-emitting region;

FIG. 2B illustrates, according to some embodiments, an exemplary system comprising a first light-emitting region comprising a first plurality of LEDs and a second light-emitting region comprising a second plurality of LEDs;

FIG. 3A illustrates an exemplary LED die, according to some embodiments;

FIG. 3B illustrates an exemplary LED comprising a packaged LED die, according to some embodiments;

FIG. 3C illustrates an exemplary LED die comprising a first flat conformal wavelength-converting layer, according to some embodiments;

FIG. 3D illustrates an exemplary LED die comprising a first flat conformal wavelength-converting layer and a second flat wavelength-converting layer, according to some embodiments;

FIG. 3E illustrates an exemplary LED die comprising a first curved conformal wavelength-converting layer comprising a first wavelength-converting material, according to some embodiments;

FIG. 3F illustrates an exemplary LED die comprising a first curved conformal wavelength-converting layer comprising a first wavelength-converting material and a second wavelength-converting material, according to some embodiments;

FIG. 3G illustrates an exemplary LED comprising a remote wavelength-converting layer, according to some embodiments;

FIG. 3H illustrates an exemplary LED comprising a first dispensed wavelength-converting material, according to some embodiments;

FIG. 3I illustrates an exemplary LED comprising a first dispensed wavelength-converting material and a second dispensed wavelength-converting material, according to some embodiments;

FIG. 4A illustrates, according to some embodiments, a chip-on-board package comprising circular regions;

FIG. 4B illustrates, according to some embodiments, a chip-on-board package comprising striped regions;

FIG. 4C illustrates, according to some embodiments, a chip-on-board package comprising striped regions and electrodes in electrical communication with each striped region; and

FIG. 5 illustrates an exemplary system comprising a plurality of surface-mounted LED packages, according to some embodiments.

### DETAILED DESCRIPTION

Aspects of the present disclosure relate to systems for emitting light (e.g., substantially white light) with tunable circadian effects and substantially consistent color characteristics, and methods of making and/or operating the same. Certain embodiments described herein are systems comprising a plurality of light-emitting regions configured to emit light having certain circadian effects (e.g., melanopic ratio) and certain color characteristics (e.g., corrected color temperature (CCT), color rendering index (CRI)). According to some embodiments, the difference between the circadian effects of the light-emitting regions may be relatively large, and the difference between the color characteristics of the light-emitting regions may be relatively small. Each light-emitting region may comprise one or more light-emitting diodes (LEDs), each of which may be associated with one or more wavelength-converting materials (e.g., phosphors).

In some cases, it may be advantageous for a system to be configured to emit light (e.g., substantially white light) with tunable circadian effects. For example, it may be desirable for a system to emit light having one effect on the circadian system (e.g., suppressing melatonin synthesis) at one time (e.g., during the day) and a different effect on the circadian system (e.g., stimulating melatonin synthesis) at a different time (e.g., in the evening). In some instances, such a system may be tuned to emit light in a pattern that mimics a human body's natural circadian rhythm, which may be associated with health benefits. For example, a light-emitting system that mimics human circadian rhythm may avoid prolonged mistimed circadian stimulation, which may negatively impact human health and increase risk for cancer and other illnesses. In other instances, a light-emitting system may be tuned to elicit unnatural biological responses (e.g., inducing sleep onset during the day and wakefulness at night). Such a system may, for example, be useful for night shift employees.

One measure of the effect of emitted light on circadian rhythm is the melanopic ratio. The melanopic ratio of a light source (e.g., a system for emitting light) can be calculated according to the following equation:

$$Melanopic Ratio = K \frac{{\int }_{380}^{780} C ( λ ) P ( λ ) d λ}{{\int }_{380}^{780} V ( λ ) P ( λ ) d λ} \qquad ( 1 )$$

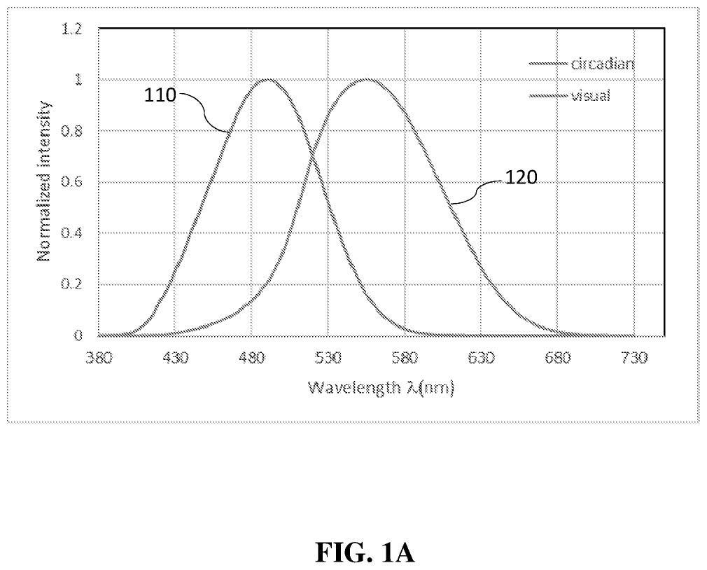

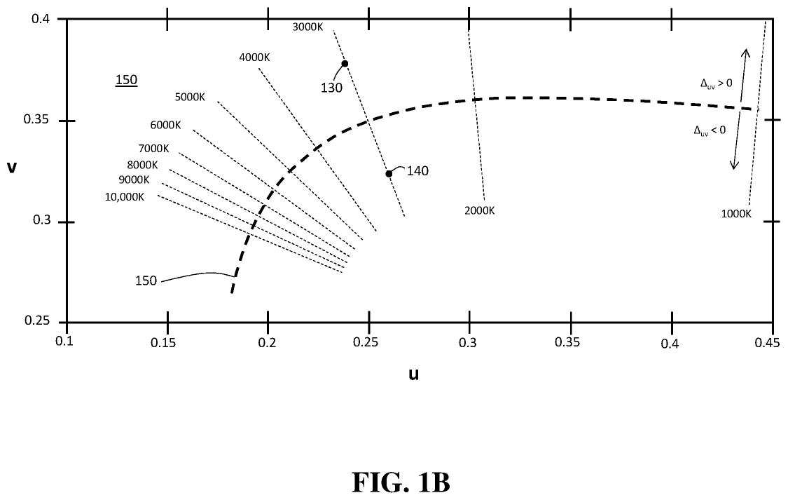

In Equation (1), P(λ) refers to the spectral power distribution (SPD) of the light emitted by the light source, C(λ) refers to the circadian action function (e.g., curve 110 in FIG. 1A), and V(λ) refers to the visual efficiency function (e.g., curve 120 in FIG. 1A). A person of ordinary skill in the art would understand that curve 110 and curve 120 in FIG. 1A, which plot data from Table L2 of the WELL Building Standard published by the International Well Building Institute (https://standard.wellcertified.com/tables#melanopicRatio), represent an exemplary circadian action function and an exemplary visual efficiency function, respectively. Circadian action function C(λ) may encompass other melanopic sensitivity functions (e.g., functions representing the wavelengths to which melanopsin-containing ipRGCs are sensitive), and visual efficiency function V(λ) may represent other photopic luminosity functions (e.g., functions representing the wavelengths to which certain rods and/or cones are sensitive).

The SPD of light emitted by a system represents the power of emitted light per unit area at each wavelength. The SPD of light emitted by a system may be measured according to any method known in the art, such as by a spectroradiometer. In Equation (1), the numerator is the SPD of a light-emitting system weighted according to the circadian action function C(λ) over the spectrum of visible light (e.g., 380 nm to 780 nm). The numerator may provide a measure of the effect of the emitted light on circadian rhythm (e.g., based on the sensitivity of melanopsin-containing ipRGCs). In Equation (1), the denominator is the SPD of the light-emitting system weighted according to the visual efficiency function V(λ) over the spectrum of visible light (i.e., 380 nm to 780 nm). The denominator may provide a measure of the amount of emitted light perceived by human vision. Generally, light having a higher melanopic ratio is associated with increased alertness and/or arousal, while light having a lower melanopic ratio is associated with increased sleepiness. In some cases, it may be desirable for a system to emit light having a relatively high melanopic ratio at a certain time of day (e.g., during the morning or afternoon) and a relatively low melanopic ratio at a different time of day (e.g., in the evening).

In some cases, it may be advantageous for light having tunable circadian effects to have substantially consistent color characteristics (e.g., CCT values, CRI values). In conventional light sources with tunable circadian effects, different circadian effects are often associated with widely different CCT values; for example, light having a first melanopic ratio may have a CCT value of 2000 K, while light having a second melanopic ratio may have a CCT value of 6500 K. In some cases, users may consider this variance in the perceived color of emitted light to be undesirable. Additionally, conventional light sources with tunable circadian effects often have relatively low CRI values. Since a low CRI value generally indicates a low quality of light, this may result in a sub-optimal user experience. In contrast, a system emitting light with substantially consistent color characteristics may provide an improved user experience (e.g., by reducing disruption and providing uniformly high quality).

One widely used measure of color quality is correlated color temperature (CCT). CCT generally refers to a metric for characterizing the color appearance of non-blackbody light emitters (e.g., LEDs) with respect to an ideal blackbody radiator (i.e., a body that absorbs radiation in all frequencies). The CCT of light emitted from a given system may be determined by plotting the chromaticity of the emitted light (i.e., u and v coordinates) on an International Commission on Illumination (CIE) chromaticity diagram (e.g., a CIE 1960 chromaticity diagram) and determining the corresponding point on the blackbody locus that is closest to the plotted point (e.g., by constructing a line segment that is perpendicular to the blackbody locus and passes through the plotted chromaticity point). Those of ordinary skill in the art are familiar with the CIE 1960 chromaticity diagram, which is a two-dimensional plot of the mathematically-defined CIE 1960 color space. One of ordinary skill in the art would be capable of determining the chromaticity of a given light output by, for example, measuring a spectrum of sufficient fidelity over the relevant wavelength range using a spectroradiometer and applying known algebraic equations. Such methods are described, for example, in the document CIE 15-2004, which is incorporated herein by reference in its entirety for all purposes. Those of ordinary skill in the art are also familiar with the blackbody locus, which is a curve corresponding to the chromaticity of radiation emitted by an ideal blackbody radiator over a range of temperatures. The blackbody locus may be computed by using the well-known Planckian formula for the emitted spectrum of an ideal blackbody radiator of a given temperature.

As an illustrative example, FIG. 1B shows a CIE 1960 chromaticity diagram with ten iso-CCT lines (i.e., lines along which all points have the same CCT value) constructed perpendicular to blackbody locus 150. In FIG. 1B, light with a chromaticity corresponding to point 130 or point 140 would have a CCT value of 3000 Kelvin (K) since points 130 and 140 are on the iso-CCT line for the color temperature of 3000 K. In general, lower CCT values are referred to as “warm,” while higher CCT values are referred to as “cool.”

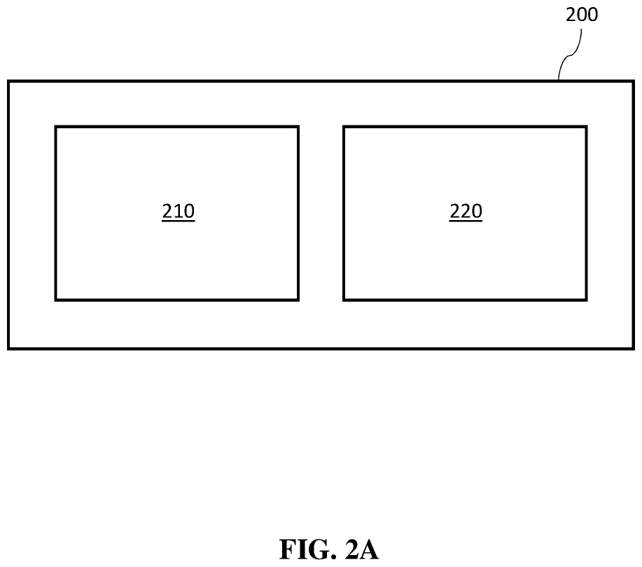

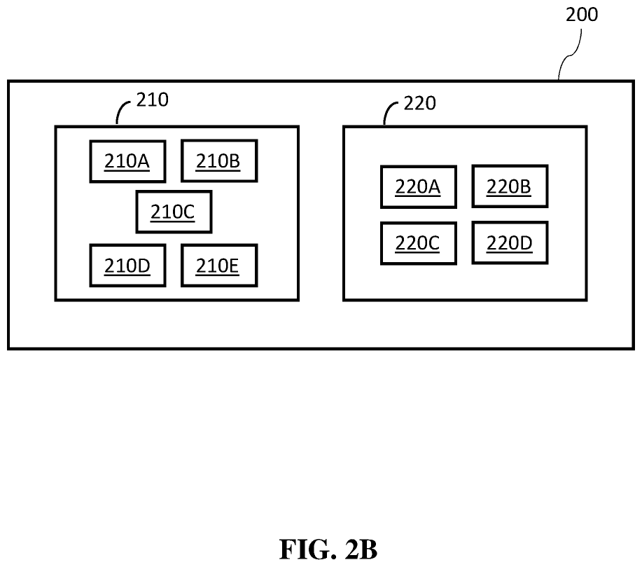

According to some embodiments, a light-emitting system comprises a plurality of light-emitting regions, where light emitted from the plurality of light-emitting regions has substantially different circadian effects and substantially similar color characteristics. As one illustrative, non-limiting embodiment, FIG. 2A shows exemplary light-emitting system 200 comprising first light-emitting region 210 and second light-emitting region 220. First light-emitting region 210 and second light-emitting region 220 may be positioned in any suitable configuration on any suitable substrate (e.g., a printed circuit board, a semiconductor wafer). In some embodiments, first light-emitting region 210 and/or second light-emitting region 220 may be configured to emit substantially white light. Light emitted from first light-emitting region 210 has a first melanopic ratio, a first CCT value, and a first CRI value, and light emitted from second light-emitting region 220 has a second melanopic ratio, a second CCT value, and a second CRI value. In some embodiments, the absolute value of the difference between the first melanopic ratio and the second melanopic ratio is relatively large (e.g., at least 0.1). In contrast, in some embodiments, the absolute value of the difference between the first CCT value and the second CCT value is relatively small (e.g., 1000 K or less). In some instances, the absolute value of the difference between the first CRI value and the second CRI value is relatively small (e.g., less than 10). In some embodiments, first light-emitting region 210 and second light-emitting region 220 are individually controlled to produce a combined output of light having varying circadian effects and/or color characteristics.

In operation, current may be directed to flow to first light-emitting region 210 during a first time period. During the first time period, light having the first melanopic ratio, the first CCT value, and the first CRI value may be emitted. In some cases, current may be directed to flow to second light-emitting region 220 instead of first light-emitting region 210 during a second time period. During the second time period, light having the second melanopic ratio, the second CCT value, and the second CRI value may be emitted. In certain instances, current may be directed to flow to first light-emitting region 210 and second light-emitting region 220 at the same time, which may result in emission of light having an intermediate melanopic ratio, CCT value, and/or CRI value.

According to some embodiments, at least two light-emitting regions of a light-emitting system have melanopic ratios that are relatively far apart. That is, in some instances, the absolute value of the difference between a first melanopic ratio of a first light-emitting region and a second melanopic ratio of a second light-emitting region is relatively large. In certain embodiments, the absolute value of the difference between a first melanopic ratio of a first light-emitting region and a second melanopic ratio of a second light-emitting region is at least 0.1, at least 0.2, at least 0.3, at least 0.4, at least 0.5, at least 0.6, at least 0.7, at least 0.8, at least 0.9, or at least 1.0. In some embodiments, the absolute value of the difference between a first melanopic ratio of a first light-emitting region and a second melanopic ratio of a second light-emitting region is in a range of 0.1 to 1.0, 0.2 to 1.0, 0.3 to 1.0, 0.4 to 1.0, 0.5 to 1.0, 0.6 to 1.0, 0.7 to 1.0, 0.8 to 1.0, or 0.9 to 1.0.

In some embodiments, each light-emitting region of a light-emitting system has a melanopic ratio that is relatively far apart from the melanopic ratio(s) of every other light-emitting region of the light-emitting system. Accordingly, in certain embodiments, the absolute value of the minimum difference between the melanopic ratios of the light-emitting regions of a light-emitting system is at least 0.1, at least 0.2, at least 0.3, at least 0.4, at least 0.5, at least 0.6, at least 0.7, at least 0.8, at least 0.9, or at least 1.0. In some embodiments, the absolute value of the minimum difference between the melanopic ratios of the light-emitting regions of a light-emitting system is in a range of 0.1 to 1.0, 0.2 to 1.0, 0.3 to 1.0, 0.4 to 1.0, 0.5 to 1.0, 0.6 to 1.0, 0.7 to 1.0, 0.8 to 1.0, or 0.9 to 1.0.

In certain embodiments, at least one light-emitting region (e.g., a first light-emitting region) of a light-emitting system has a relatively low melanopic ratio. In some instances, at least one light-emitting region has a melanopic ratio of 0.9 or less, 0.8 or less, 0.7 or less, 0.6 or less, 0.5 or less, 0.4 or less, 0.3 or less, 0.2 or less, or 0.1 or less.

In certain cases, at least one light-emitting region has a melanopic ratio in a range from 0.0 to 0.4, 0.0 to 0.3, 0.0 to 0.2, 0.0 to 0.1, 0.1 to 0.4, 0.1 to 0.3, 0.1 to 0.2, 0.2 to 0.4, 0.3 to 0.5, 0.4 to 0.8, 0.4 to 0.6, 0.6 to 1.0, 0.6 to 0.8, 0.7 to 1.1, 0.7 to 0.9, 0.8 to 1.2, or 0.8 to 0.9.

In some embodiments, at least one light-emitting region (e.g., a second light-emitting region) of a light-emitting system has a relatively high melanopic ratio. In some instances, at least one light-emitting region has a melanopic ratio of at least 0.3, at least 0.4, at least 0.5, at least 0.6, at least 0.7, at least 0.8, at least 0.9, at least 1.0, at least 1.1, at least 1.2, at least 1.3, at least 1.4, or at least 1.5. In some embodiments, at least one light-emitting region has a melanopic ratio in a range from 0.3 to 1.0, 0.3 to 1.5, 0.4 to 0.6, 0.4 to 1.0, 0.4 to 1.5, 0.5 to 0.7, 0.5 to 1.0, 0.5 to 1.2, 0.5 to 1.5, 0.6 to 0.8, 0.6 to 1.0, 0.6 to 1.5, 0.7 to 0.9, 0.7 to 1.0, 0.7 to 1.5, 0.8 to 1.0, 0.8 to 1.5, 0.9 to 1.1, 0.9 to 1.5, 1.0 to 1.2, 1.0 to 1.5, 1.1 to 1.2, or 1.1 to 1.5.

According to some embodiments, at least two light-emitting regions of a light-emitting system have CCT values that are relatively similar. That is, in some instances, the absolute value of the difference between a first CCT value of a first light-emitting region and a second CCT value of a second light-emitting region is relatively small. In certain embodiments, the absolute value of the difference between a first CCT value of a first light-emitting region and a second CCT value of a second light-emitting region is 1000 K or less, 900 K or less, 800 K or less, 700 K or less, 600 K or less, 500 K or less, 400 K or less, 300 K or less, 200 K or less, 100 K or less, 50 K or less, or 10 K or less. In some embodiments, the absolute value of the difference between a first CCT value of a first light-emitting region and a second CCT value of a second light-emitting region is between 0 K and 1000 K, between 0 K and 900 K, between 0 K and 800 K, between 0 K and 700 K, between 0 K and 600 K, between 0 K and 500 K, between 0 K and 400 K, between 0 K and 300 K, between 0 K and 200 K, between 0 K and 100 K, between 0 K and 50 K, or between 0 K and 10 K.

In some embodiments, all light-emitting regions of a light-emitting system have relatively similar CCT values. In some embodiments, the absolute value of the maximum difference between the CCT values of the light-emitting regions of a light-emitting system is 1000 K or less, 900 K or less, 800 K or less, 700 K or less, 600 K or less, 500 K or less, 400 K or less, 300 K or less, 200 K or less, 100 K or less, 50 K or less, or 10 K or less. In some embodiments, the absolute value of the maximum difference between the CCT values of the light-emitting regions of a light-emitting system is between 0 K and 1000 K, between 0 K and 900 K, between 0 K and 800 K, between 0 K and 700 K, between 0 K and 600 K, between 0 K and 500 K, between 0 K and 400 K, between 0 K and 300 K, between 0 K and 200 K, between 0 K and 100 K, between 0 K and 50 K, or between 0 K and 10 K.

In some embodiments, at least one light-emitting region (e.g., a first light-emitting region, a second light-emitting region) of a light-emitting system is configured to emit relatively “warm” light. In certain embodiments, at least one light-emitting region has a CCT value of 3000 K or less, 2000 K or less, or 1000 K or less. In some embodiments, at least one light-emitting region has a CCT value in a range from 0 K to 1000 K, 0 K to 2000 K, 0 K to 3000 K, 1000 K to 2000 K, 1000 K to 3000 K, or 2000 K to 3000 K.

In some embodiments, every light-emitting region of a light-emitting system is configured to emit relatively “warm” light. In certain embodiments, every light-emitting region of a light-emitting system has a CCT value of 3000 K or less, 2000 K or less, or 1000 K or less. In some embodiments, every light-emitting region of a light-emitting system has a CCT value in a range from 0 K to 1000 K, 0 K to 2000 K, 0 K to 3000 K, 1000 K to 2000 K, 1000 K to 3000 K, or 2000 K to 3000 K.

In some embodiments, at least one light-emitting region (e.g., a first light-emitting region, a second light-emitting region) of a light-emitting system is configured to emit relatively “cool” light. In certain embodiments, at least one light-emitting region of a light-emitting system has a CCT value of at least 3500 K, at least 4000 K, at least 5000 K, at least 6000 K, or at least 6500 K, at least 7000 K, at least 8000 K, or at least 9000 K. In some embodiments, at least one light-emitting region of a light-emitting system has a CCT value in a range from 3500 K to 5000 K, 3500 K to 6000 K, 3500 K to 6500 K, 3500 K to 7000 K, 3500 K to 8000 K, 3500 K to 9000 K, 4000 K to 6000 K, 4000 K to 6500 K, 5000 K to 6000 K, or 5000 K to 6500 K.

In some embodiments, every light-emitting region of a light-emitting system is configured to emit relatively “cool” light. In certain embodiments, every light-emitting region of a light-emitting system has a CCT value of at least 3500 K, at least 4000 K, at least 5000 K, at least 6000 K, or at least 6500 K, at least 7000 K, at least 8000 K, or at least 9000 K. In some embodiments, every light-emitting region of a light-emitting system has a CCT value in a range from 3500 K to 5000 K, 3500 K to 6000 K, 3500 K to 6500 K, 3500 K to 7000 K, 3500 K to 8000 K, 3500 K to 9000 K, 4000 K to 6000 K, 4000 K to 6500 K, 5000 K to 6000 K, or 5000 K to 6500 K.

In some cases, at least one light-emitting region (e.g., a first light-emitting region, a second light-emitting region) that is configured to emit light having certain CCT values is also configured to emit light having a range of melanopic ratios. In certain embodiments, a plurality of light-emitting regions (and, in some cases, all light-emitting regions) of a light-emitting system are configured to emit light having a CCT value of 6000 K to 7000 K, and at least one light-emitting region (e.g., a first light-emitting region) of the plurality of light-emitting regions is configured to emit light having a melanopic ratio of 0.8 to 1.0 or 0.8 to 0.9, and at least one light-emitting region (e.g., a second light-emitting region) of the plurality of light-emitting regions is configured to emit light having a melanopic ratio of 1.0 to 1.2 or 1.1 to 1.2. In certain embodiments, a plurality of light-emitting regions (and, in some cases, all light-emitting regions) of a light-emitting system are configured to emit light having a CCT value of 5000 K to 6000 K, and at least one light-emitting region (e.g., a first light-emitting region) of the plurality of light-emitting regions is configured to emit light having a melanopic ratio of 0.7 to 0.9, and at least one light-emitting region (e.g., a second light-emitting region) of the plurality of light-emitting regions is configured to emit light having a melanopic ratio of 1.0 to 1.2. In certain embodiments, a plurality of light-emitting regions (and, in some cases, all light-emitting regions) of a light-emitting system are configured to emit light having a CCT value of 4000 K to 5000 K, and at least one light-emitting region (e.g., a first light-emitting region) of the plurality of light-emitting regions is configured to emit light having a melanopic ratio of 0.6 to 0.8, and at least one light-emitting region (e.g., a second light-emitting region) of the plurality of light-emitting regions is configured to emit light having a melanopic ratio of 0.9 to 1.1. In certain embodiments, a plurality of light-emitting regions (and, in some cases, all light-emitting regions) of a light-emitting system are configured to emit light having a CCT value of 3000 K to 4000 K, and at least one light-emitting region (e.g., a first light-emitting region) of the plurality of light-emitting regions is configured to emit light having a melanopic ratio of 0.4 to 0.6, and at least one light-emitting region (e.g., a second light-emitting region) of the plurality of light-emitting regions is configured to emit light having a melanopic ratio of 0.7 to 0.9. In certain embodiments, a plurality of light-emitting regions (and, in some cases, all light-emitting regions) of a light-emitting system are configured to emit light having a CCT value of 2000 K to 3000 K, and at least one light-emitting region (e.g., a first light-emitting region) of the plurality of light-emitting regions is configured to emit light having a melanopic ratio of 0.3 to 0.5, and at least one light-emitting region (e.g., a second light-emitting region) of the plurality of light-emitting regions is configured to emit light having a melanopic ratio of 0.5 to 0.7.

According some embodiments, at least one light-emitting region of a light-emitting system has a relatively high color rendering index (CRI) value. CRI refers to a quantitative measure of the accuracy with which a light source renders color of illuminated objects as compared to an ideal blackbody radiator, where a higher CRI value indicates higher accuracy (i.e., more natural rendering of colors). For example, sunlight has a CRI of 100, which is the highest possible value. CRI may be measured according to any method known in the art, such as by the 1995 CIE Method of Measuring and Specifying Colour Rendering Properties of Light Sources.

In some embodiments, at least one light-emitting region of a light-emitting system has a CRI value of at least 60, at least 70, at least 80, at least 85, at least 90, at least 95, at least 99, or about 100. In some embodiments, at least one light-emitting region of a light-emitting system has a CRI value in a range from 60 to 100, 70 to 100, 80 to 100, 85 to 100, 90 to 100, or 95 to 100.

In some embodiments, every light-emitting region of a light-emitting system has a CRI value of at least 60, at least 70, at least 80, at least 85, at least 90, at least 95, at least 99, or about 100. In some embodiments, every light-emitting region of a light-emitting system has a CRI value in a range from 60 to 100, 70 to 100, 80 to 100, 85 to 100, 90 to 100, or 95 to 100.

According to some embodiments, at least two light-emitting regions of a light-emitting system have relatively similar CRI values. That is, in some instances, the absolute value of the difference between a first CRI value of a first light-emitting region and a second CRI value of a second light-emitting region is relatively small. In certain embodiments, the absolute value of the difference between a first CRI value of a first light-emitting region and a second CRI value of a second light-emitting region is 20 or less, 15 or less, 10 or less, 5 or less, 2 or less, 1 or less, or about 0. In some embodiments, the absolute value of the difference between a first CRI value of a first light-emitting region and a second CRI value of a second light-emitting region is in a range from 0 to 20, 0 to 15, 0 to 10, or 0 to 5.

In some embodiments, all light-emitting regions of a light-emitting system have relatively similar CRI values. In some embodiments, the absolute value of the maximum difference between the CRI values of the light-emitting regions of a light-emitting system is 20 or less, 15 or less, 10 or less, 5 or less, 2 or less, 1 or less, or about 0. In some embodiments, the absolute value of the maximum difference between the CRI values of the light-emitting regions of a light-emitting system is in a range from 0 to 20, 0 to 15, 0 to 10, or 0 to 5.

Light-emitting systems described herein may have any combination of the above-described characteristics. In some embodiments, at least two light-emitting regions (e.g., a first light-emitting region, a second light-emitting region) of a light-emitting system have melanopic ratios that are relatively far apart and CCT values that are relatively close together. In certain instances, the absolute value of the difference between a first CCT value of the first light-emitting region and a second CCT value of the second light-emitting region (and, in some instances, between CCT values of each pair of light-emitting regions) is between 0 K and 500 K, between 0 K and 400 K, between 0 K and 300 K, between 0 K and 200 K, between 0 K and 100 K, or between 0 K and 50 K, and the absolute value of the difference between a first melanopic ratio of the first light-emitting region and a second melanopic ratio of the second light-emitting region (and, in some instances, between the melanopic ratios of each pair of light-emitting regions) is at least 0.1, at least 0.2, at least 0.3, or at least 0.4. In some instances, the absolute value of the difference between a first CCT value of the first light-emitting region and a second CCT value of the second light-emitting region (and, in some instances, between the CCT values of each pair of light-emitting regions) is between 500 K and 1000 K, between 500 K and 900 K, between 500 K and 800 K, between 500 K and 700 K, or between 500 K and 600 K, and the absolute value of the difference between the first melanopic ratio of the first light-emitting region and the second melanopic ratio of the second light-emitting region (and, in some instances, between the melanopic ratios of each pair of light-emitting regions) is at least 0.5, at least 0.6, at least 0.7, at least 0.8, at least 0.9, or at least 1.0.

In some embodiments, at least two light-emitting regions (e.g., a first light-emitting region, a second light-emitting region) of a light-emitting system have melanopic ratios that are relatively far apart and CRI values that are relatively close together. In certain instances, the absolute value of the difference between a first CRI value of the first light-emitting region and a second CRI value of the second light-emitting region (and, in some instances, between CRI values of each pair of light-emitting regions) is between 0 and 20, between 0 and 15, between 0 and 10, or between 0 and 5, and the absolute value of the difference between a first melanopic ratio of the first light-emitting region and a second melanopic ratio of the second light-emitting region (and, in some instances, between the melanopic ratios of each pair of light-emitting regions) is at least 0.1, at least 0.2, at least 0.3, at least 0.4, at least 0.5, at least 0.6, at least 0.7, at least 0.8, at least 0.9, or at least 1.0.

In some embodiments, at least two light-emitting regions (e.g., a first light-emitting region, a second light-emitting region) of a light-emitting system have melanopic ratios that are relatively far apart and relatively high CRI values. In certain instances, a first CRI value of the first light-emitting region and a second CRI value of the second light-emitting region (and, in some instances, CRI values of all light-emitting regions) are each at least 60, at least 70, at least 80, at least 85, at least 90, at least 95, at least 99, or about 100, and the absolute value of the difference between a first melanopic ratio of the first light-emitting region and a second melanopic ratio of the second light-emitting region (and, in some instances, between the melanopic ratios of each pair of light-emitting regions) is at least 0.1, at least 0.2, at least 0.3, at least 0.4, at least 0.5, at least 0.6, at least 0.7, at least 0.8, at least 0.9, or at least 1.0.

In certain embodiments, at least two light-emitting regions (e.g., a first light-emitting region, a second light-emitting region) of a light-emitting system have melanopic ratios that are relatively far apart, CCT values that are relatively close together, and relatively high CRI values. In some instances, the absolute value of the difference between the first CCT value of the first light-emitting region and the second CCT value of the second light-emitting region (and, in some instances, between the CCT values of each pair of light-emitting regions) is between 0 K and 1000 K, between 0 K and 900 K, between 0 K and 800 K, between 0 K and 700 K, between 0 K and 600 K, between 0 K and 500 K, between 0 K and 400 K, between 0 K and 300 K, between 0 K and 200 K, between 0 K and 100 K, or between 0 K and 50 K, the first CRI value of the first light-emitting region and the second CRI value of the second light-emitting region (and, in some instances, CRI values of all light-emitting regions) are each at least 60, at least 70, at least 80, at least 85, at least 90, at least 95, at least 99, or about 100, and the absolute value of the difference between the first melanopic ratio of the first light-emitting region and the second melanopic ratio of the second light-emitting region (and, in some instances, between the melanopic ratios of each pair of light-emitting regions) is at least 0.1, at least 0.2, at least 0.3, at least 0.4, at least 0.5, at least 0.6, at least 0.7, at least 0.8, at least 0.9, or at least 1.0.

In some embodiments, at least one light-emitting region of a light-emitting system emits substantially white light. In certain embodiments, every light-emitting region of a light-emitting system emits substantially white light. The term “substantially white light” is generally used herein to refer to light having a chromaticity that, when plotted on the CIE 1960 chromaticity diagram, defines a Δuv value having an absolute value of less than or equal to about 0.05. One of ordinary skill in the art would understand that the Δuv value (also written as “delta(uv) value”) of a given point on the CIE 1960 chromaticity diagram corresponds to the shortest distance between the point and the blackbody locus.

In some embodiments, at least one light-emitting region of a light-emitting system is configured to emit substantially white light with a Δuv value having an absolute value of 0.02 or less, 0.01 or less, 0.005 or less, or 0.002 or less. In some embodiments, at least one light-emitting region of a light-emitting system is configured to emit substantially white light with a Δuv value in a range from 0.0 to 0.002, 0.0 to 0.005, 0.0 to 0.01, 0.0 to 0.02, 0.001 to 0.005, 0.001 to 0.01, or 0.001 to 0.02.

In some embodiments, every light-emitting region of a light-emitting system is configured to emit substantially white light with a Δuv value having an absolute value of 0.02 or less, 0.01 or less, 0.005 or less, or 0.002 or less. In some embodiments, every light-emitting region of a light-emitting system is configured to emit substantially white light with a Δuv value in a range from 0.0 to 0.002, 0.0 to 0.005, 0.0 to 0.01, 0.0 to 0.02, 0.001 to 0.005, 0.001 to 0.01, or 0.001 to 0.02.

According to some embodiments, a light-emitting system comprises a plurality of light-emitting regions. The plurality of light-emitting regions may comprise any number of light-emitting regions. In some embodiments, the plurality of light-emitting regions comprises two, three, four, five, six, seven, eight, nine, ten, or more light-emitting regions. The light-emitting regions of a light-emitting system may be positioned in any suitable arrangement. In some embodiments, the plurality of light-emitting regions may be arranged to form an array (e.g., an array with a regularly-repeating unit cell). In some embodiments, the plurality of light-emitting regions may be irregularly arranged.

In some embodiments, at least one light-emitting region of a light-emitting system comprises at least one LED. In certain cases, at least one light-emitting region comprises a plurality of LEDs. The plurality of LEDs may have any number of LEDs. In some instances, for example, the plurality of LEDs comprises at least 2, at least 3, at least 4, at least 5, at least 10, at least 15, at least 20, at least 50, or at least 100 LEDs. In some instances, the plurality of LEDs comprises between 2 and 10 LEDs, between 2 and 20 LEDs, between 2 and 50 LEDs, between 2 and 100 LEDs, between 10 and 50 LEDs, between 10 and 100 LEDs, or between 50 and 100 LEDs. In some embodiments, at least two LEDs, and in some instances all LEDs, of a plurality of LEDs of a light-emitting region emit light having substantially the same melanopic ratio, the same CCT value, and/or the same CRI value. In certain embodiments, at least 50%, at least 60%, at least 70%, at least 80%, at least 90%, at least 95%, at least 99%, or about 100% of the LEDs of the plurality of LEDs of a light-emitting region emit light having substantially the same melanopic ratio, the same CCT value, and/or the same CRI value. In some cases, the percent average deviation from the mean for melanopic ratio, CCT value, CRI value, and/or another circadian or color characteristic, is 20% or less, 15% or less, 10% or less, 5% or less, 1% or less, or about 0%. In certain instances, the percent average deviation from the mean for melanopic ratio, CCT value, CRI value, and/or another circadian or color characteristic, is between 0% and 1%, between 0% and 5%, between 0% and 10%, between 0% and 15%, or between 0% and 20%. A person of ordinary skill in the art would understand percent average deviation from the mean to be calculated according to the following equation:

$$\% average deviation = \frac{\frac{1}{N} ( {x}_{i} - \overline{x} )}{\overline{x}} \times 100$$

where N is the number of LEDs, xᵢ is the value (e.g., melanopic ratio, CCT value, CRI value) for the i^{th} LED, and x̅ is the number-weighted mean value. In embodiments where a light-emitting region comprises a plurality of LEDs, any circadian or color characteristic of the light-emitting region (e.g., melanopic ratio, CCT value, CRI value) refers to the circadian or color characteristic of the combined output of all LEDs of the plurality of LEDs of the light-emitting region.

In embodiments where a light-emitting region comprises a plurality of LEDs, the LEDs of the plurality of LEDs may be positioned in any suitable arrangement. In some embodiments, the plurality of LEDs may be arranged to form an array (e.g., an array with a regularly-repeating unit cell). In some embodiments, the plurality of LEDs may be irregularly arranged. The LEDs within a plurality of LEDs may be spaced any suitable distance apart from each other. In certain embodiments, the LEDs of a plurality of LEDs are spaced relatively close together. For example, in certain embodiments, the largest nearest-neighbor distance between any two LEDs in a plurality of LEDs is 10 cm or less, 10 mm or less, 1 mm or less, 500 μm or less, or 100 μm or less. The nearest-neighbor distance between a first LED and a second LED generally refers to the shortest distance between the edges of the first LED and the edges of the second LED. The LEDs of the plurality of LEDs may be LED chips or individually packaged LEDs.

In some embodiments, at least two light-emitting regions of a light-emitting system each comprise a plurality of LEDs. In such embodiments, each plurality of LEDs may have the same or different numbers of LEDs and the same or different arrangements of LEDs (e.g., arranged to form an array, irregularly arranged). As an illustrative, non-limiting embodiment, FIG. 2B shows first light-emitting region 210 of system 200 as comprising a first plurality of LEDs comprising five LEDs (210A, 210B, 210C, 210D, 210E) arranged in a first array. In some instances, one or more LEDs, and in some instances all LEDs, of the first plurality of LEDs emit light having the first melanopic ratio, the first CCT value, and/or the first CRI value. FIG. 2B further shows second light-emitting region 220 of system 200 as comprising a second plurality of LEDs comprising four LEDs (220A, 220B, 220C, 220D) arranged in a second array. In some instances, one or more LEDs, and in some instances all LEDs, of the second plurality of LEDs emit light having the second melanopic ratio, the second CCT value, and/or the second CRI value.

In some embodiments, a light-emitting system comprising a plurality of light-emitting regions comprises a controller in electrical communication with at least two light-emitting regions of the plurality of light-emitting regions. In some embodiments, the controller may be configured to adjust the circadian effects (e.g., melanopic ratio) of light emitted from the system. As one example, the controller may comprise a general purpose processor that is programmed to refer to a lookup table (e.g., stored in memory) such that the controller adjusts the melanopic ratio of emitted light (i.e., the melanopic ratio of the cumulative light output) according to the time of day.

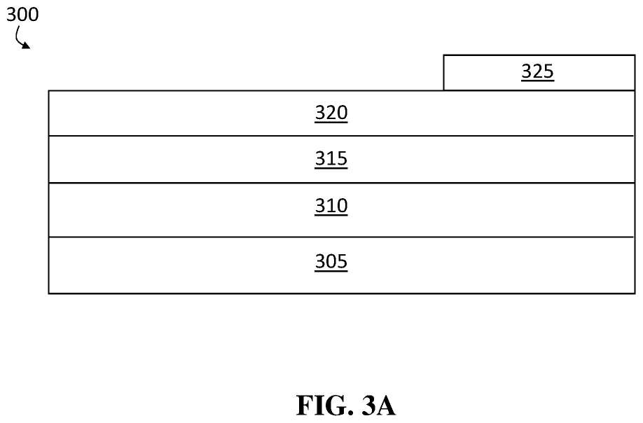

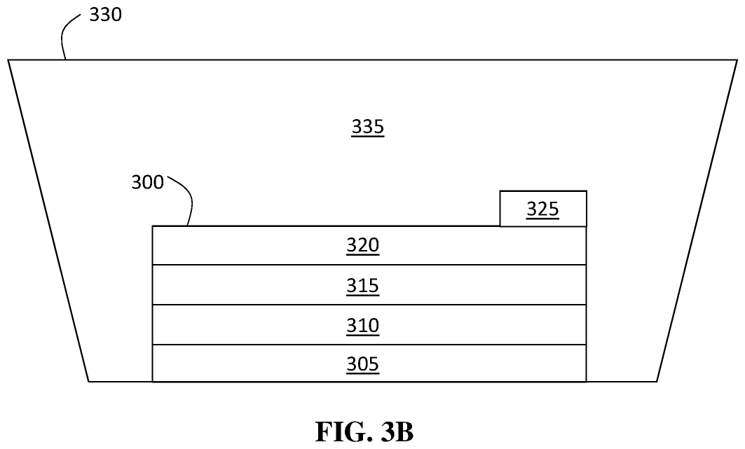

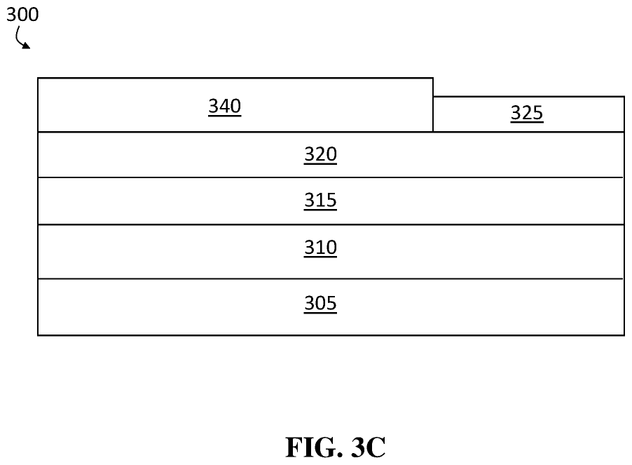

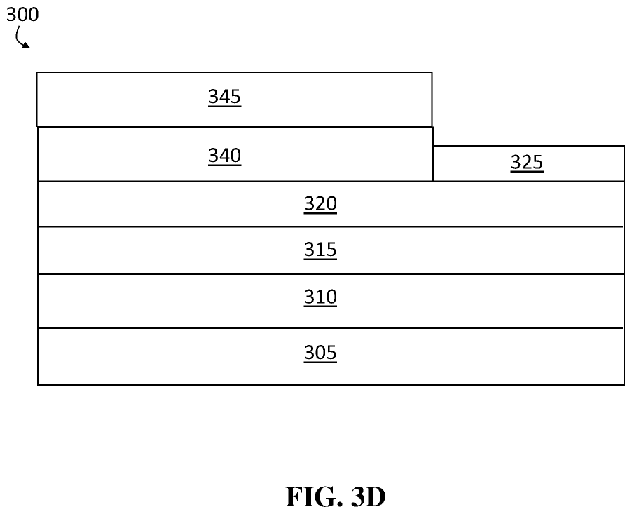

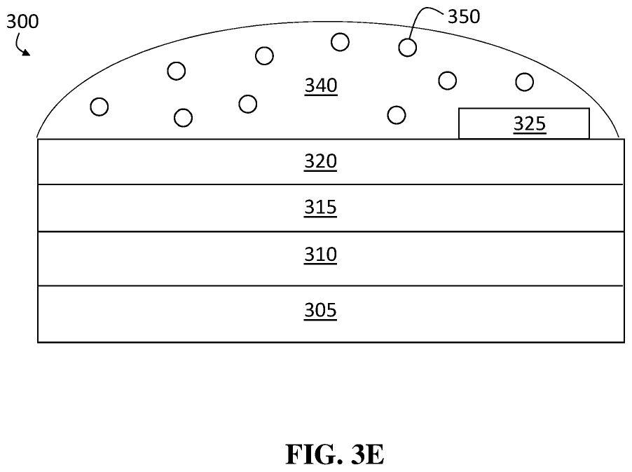

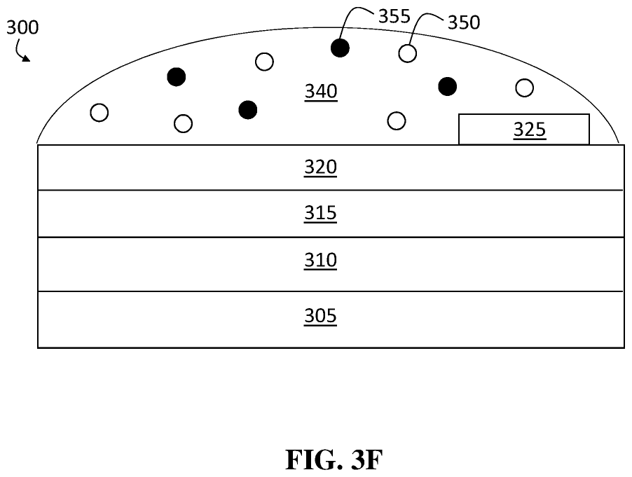

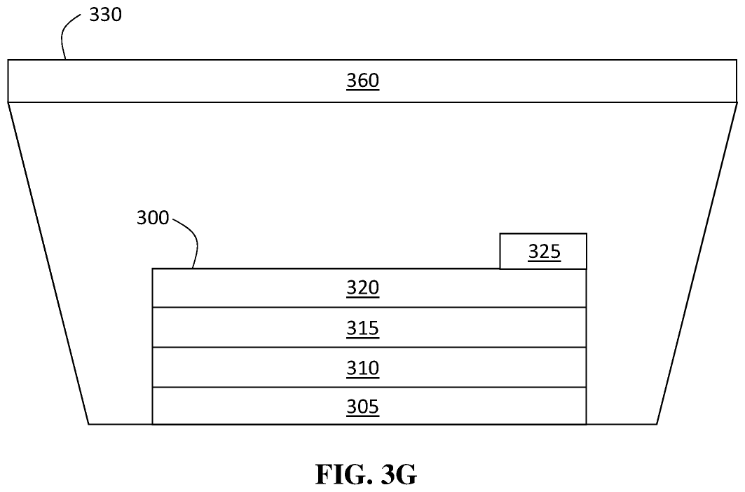

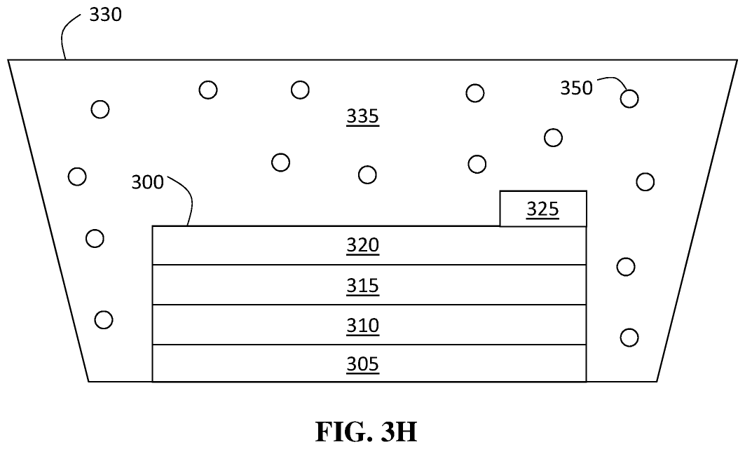

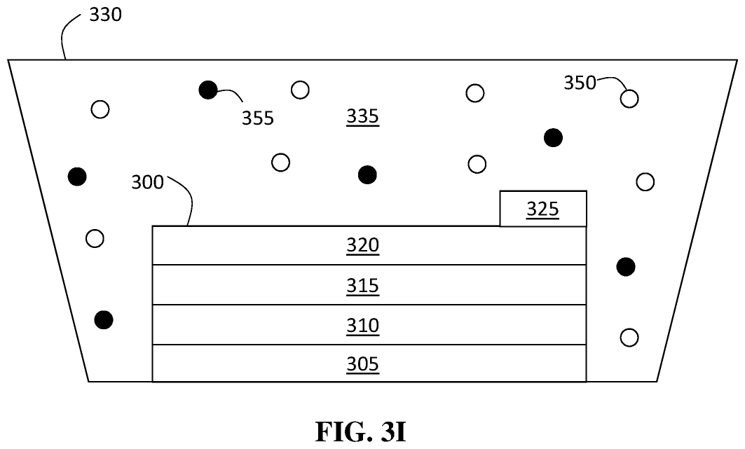

Any suitable type of LED may be used in the systems described herein. In some embodiments, the LED comprises an LED die. The LED may comprise multiple layers, at least some of which are formed of different materials. FIG. 3A is a schematic illustration of an exemplary LED die 300 that may be used in connection with the embodiments described herein. In FIG. 3A, LED die 300 comprises electrically conductive layer 305, p-doped layer(s) 310 (i.e., layer(s) doped with acceptor atoms that result in a relatively high hole concentration), light-generating region 315, n-doped layer(s) 320 (i.e., layer(s) doped with donor atoms that result in high electron concentration), and electrically conductive layer 325. As shown in FIG. 3A, light-generating region 315 may be formed between n-doped layer(s) 320 and p-doped layer(s) 310. Electrically conductive layer 305 may be in electrical communication with p-doped layer(s) 310 and may serve as a p-side contact. In certain embodiments, electrically conductive layer 305 may be in direct physical contact with p-doped layer(s) 310. Electrically conductive layer 325 may be in electrical communication with n-doped layer(s) 320 and may serve as an n-side contact. In certain embodiments, electrically conductive layer 325 may be in direct physical contact with n-doped layer(s) 320. It should be appreciated that various embodiments presented herein can also be applied to LEDs having other configurations (including organic LEDs, also referred to as OLEDs, and configurations in which the n-doped and p-doped layers are interchanged).

In operation, electrically conductive layer 305 (i.e., the p-side contact layer) can be held at a positive potential relative to electrically conduct layer 325 (i.e., the n-side contact layer), which causes electrical current to be injected into the LED. As the electrical current passes through light-generating region 315, electrons from n-doped layer 320 can combine in the light-generating region with holes from p-doped layer 310, which can cause light-generating region 315 to generate light. Light-generating region 315 can contain a multitude of point dipole radiation sources that generate light with a spectrum of wavelengths characteristic of the material from which the light-generating region is formed.

In some embodiments, the LED die may be packaged. Any suitable package configuration may be used. Non-limiting examples of suitable package configurations include lead frame or ceramic packages (e.g., lead or ceramic frames comprising one or more cavities), surface-mounted packages, and chip-on-board packages. In certain embodiments, one or more packages may be mounted on a substrate (e.g., a printed circuit board, a semiconductor wafer) according to any process known in the art (e.g., a surface-mounting process). As a non-limiting example, FIG. 3B illustrates an exemplary embodiment comprising package 330. As shown in FIG. 3B, package 330 comprises cavity 335. LED die 300 is positioned within cavity 335. In certain embodiments, one or more surfaces (e.g., side surfaces, bottom surface) of cavity 335 may be at least partially reflective.

According to some embodiments, the LED die is associated with one or more wavelength-converting materials (e.g., phosphors). The one or more wavelength-converting materials (e.g., phosphors) may be associated with the LED die according to any method known in the art. In certain embodiments, an LED die comprises one or more wavelength-converting layers comprising one or more wavelength-converting materials (e.g., phosphors). In some embodiments, the one or more wavelength-converting layers may be associated with more than one LED die. For example, the one or more wavelength-converting layers may be associated with at least 2, at least 3, at least 5, at least 10, or at least 100 LED dies. In some embodiments, an LED package comprises a cavity, and a mixture comprising one or more wavelength-converting materials (e.g., phosphors) is dispensed in at least a portion of the cavity. In some embodiments, the cavity may comprise more than one LED die. For example, the cavity may comprise at least 2, at least 3, at least 5, at least 10, or at least 100 LED dies.

In embodiments comprising one or more wavelength-converting layers, the layers may be positioned at any distance from the LED die. In some embodiments, at least one wavelength-converting layer is substantially conformal. A person of ordinary skill in the art would understand a substantially conformal layer to refer to a layer that is at least partially (and, in some cases, fully) in direct physical contact with a surface of the LED die. A substantially conformal layer may advantageously position a wavelength-converting material in close proximity to a light-generating region of an LED die. In some embodiments, at least one wavelength-converting layer is a remote layer. A person of ordinary skill in the art would understand a remote layer to refer to a layer that is not in direct physical contact with a surface of the LED die. A remote wavelength-converting layer may be particularly desirable in light-emitting systems comprising high-efficiency LEDs and/or involving low-power applications (e.g., displays).

In embodiments comprising one or more wavelength-converting layers, the one or more layers may have any suitable shape. In some cases, a surface (e.g., a top surface, a side surface) of the one or more wavelength-converting layers may be substantially flat or substantially curved. The one or more wavelength-converting layers may also have any suitable thickness. In some cases, for a given concentration of wavelength-converting materials, a thicker layer may result in warmer emitted light, while a thinner layer may result in cooler emitted light. Each wavelength-converting layer of the one or more wavelength-converting layers may comprise any number of wavelength-converting materials (e.g., one wavelength-converting material, two wavelength-converting materials, three-wavelength-converting materials, etc.). In certain embodiments, the one or more wavelength-converting layers comprise two or more wavelength-converting layers. In some such embodiments, the two or more wavelength-converting layers may comprise multiple layers comprising the same wavelength-converting material and/or multiple layers comprising different wavelength-converting materials. The layers of wavelength-converting material may be deposited using one or more masking steps, with or without subsequent removal of the masks between the application of different layers.

FIG. 3C illustrates an exemplary LED die comprising a first wavelength-converting layer. In FIG. 3C, first wavelength-converting layer 340 comprising a first wavelength-converting material (e.g., a first phosphor) is positioned on n-doped layer 320 such that at least a portion of the light generated within light-generating region 315 is absorbed by the first wavelength-converting material of the first wavelength-converting layer 340 and converted into light comprising wavelengths different from those generated within light-generating region 315. In some such embodiments, light-generating region 315 may be configured to generate non-white light, and the first wavelength-converting material may be configured to produce substantially white light from the non-white light. In the embodiment illustrated in FIG. 3C, first wavelength-converting layer 340 is substantially conformal (e.g., at least a portion of first wavelength-converting layer 340 is in direct physical contact with at least a portion of LED die 300). Additionally, in FIG. 3C, first wavelength-converting layer 340 has a substantially flat top surface. In some embodiments, first wavelength-converting layer 340 comprises one or more wavelength-converting materials (e.g., a second phosphor, a third phosphor) in addition to the first wavelength-converting material.

In some embodiments, the LED further comprises a second wavelength-converting layer comprising a second wavelength-converting material (e.g., a second phosphor). FIG. 3D illustrates an exemplary LED die comprising a second wavelength-converting layer 345. In FIG. 3D, second wavelength-converting layer 345 is positioned on first wavelength-converting layer 340 such that at least a portion of the light emitted from first wavelength-converting layer 340 is absorbed by the second wavelength-converting material of the second wavelength-converting layer 345 and converted into light comprising wavelengths different from those emitted from first wavelength-converting layer 340. In some such embodiments, light-generating region 315 and/or first wavelength-converting layer 340 may be configured to generate non-white light, and the second wavelength-converting material may be configured to produce substantially white light when combined with the light emitted from light-generating region 315 and/or first wavelength-converting layer 340. In the embodiment illustrated in FIG. 3D, first wavelength-converting layer 340 and second wavelength-converting layer 345 each have a substantially flat top surface. In some embodiments, second wavelength-converting layer comprises one or more wavelength-converting materials (e.g., a third phosphor, a fourth phosphor) in addition to the second wavelength-converting material.

In some embodiments, one or more surfaces (e.g., a top surface, a side surface) of a wavelength-converting layer are substantially curved. As an illustrative example, in FIG. 3E, first wavelength-converting layer 340 has a substantially curved top surface. In certain embodiments, one or more surfaces of a wavelength-converting layer may be positioned to cover at least a portion of a side surface of LED die 300. Such embodiments may be particularly desirable in light-emitting systems comprising one or more LEDs in which at least some light is emitted via a side surface of the LED (e.g., lateral LEDs). In some such embodiments, one or more surfaces (e.g., side surfaces) of a wavelength-converting layer positioned to cover at least a portion of a side surface of an LED may be substantially curved.

FIG. 3E shows LED die 300 comprising wavelength-converting layer 340 having a substantially curved top surface. In some instances, substantially curved wavelength-converting layer 340 comprises a first wavelength-converting material (e.g., a first phosphor). As a non-limiting, illustrative example, FIG. 3E shows a substantially curved wavelength-converting layer 340 comprising particles 350 of a first wavelength-converting material. In some instances, substantially curved wavelength-converting layer 340 comprises a first wavelength-converting material and a second wavelength-converting material. As a non-limiting, illustrative example, FIG. 3F shows a substantially curved wavelength-converting layer 340 comprising particles 350 of a first wavelength-converting material and particles 355 of a second wavelength-converting material. In some embodiments, the first wavelength-converting material is different from the second wavelength-converting material. In certain embodiments, for example, at least a portion of light emitted from the first wavelength-converting material is absorbed by the second wavelength-converting material and converted into light having a different wavelength than the light emitted from the first wavelength-converting material. In some embodiments, substantially curved wavelength-converting layer 340 comprises three, four, five, or more different wavelength-converting materials.

In some embodiments, a wavelength-converting layer is a remote layer (i.e., not in direct physical contact with a surface of an LED die). FIG. 3G illustrates an exemplary embodiment comprising a remote wavelength-converting layer 360 positioned on a top surface of LED package 330. Remote wavelength-converting layer 360 may comprise any suitable number of wavelength-converting materials.

In certain embodiments, a mixture comprising one or more wavelength-containing materials (e.g., phosphors) is dispensed in at least a portion of an LED package (e.g., a cavity in which an LED die is positioned). The mixture may comprise any suitable encapsulant, including, but not limited to, a silicone and/or an epoxy. FIG. 3H illustrates an exemplary LED comprising a dispensed wavelength-converting material. In FIG. 3H, LED package 330 contains LED die 300 positioned within cavity 335. In the embodiment shown in FIG. 3H, cavity 335 is filled with a mixture comprising particles 350 of a first wavelength-converting material (e.g., a first phosphor). Particles 350 may be dispersed in any suitable encapsulant (e.g., a silicone, an epoxy).

In some embodiments, two or more wavelength-converting materials (e.g., phosphors) are dispensed in an LED. For example, FIG. 3I illustrates an exemplary LED comprising two wavelength-converting materials. Like the illustrative embodiment shown in FIG. 3H, the embodiment shown in FIG. 3I comprises LED package 330 containing LED die 300 positioned within cavity 335. The remainder of cavity 335 is filled with a mixture comprising particles 350 of a first wavelength-converting material (e.g., a first phosphor) and particles 355 of a second wavelength-converting material (e.g., a second phosphor). Particles 350 and 355 may be dispersed in any suitable encapsulant (e.g., a silicone, an epoxy).

In some embodiments, p-doped layer 310 and/or n-doped layer 320 comprise a semiconductor material. Non-limiting examples of suitable semiconductor materials include III-V semiconductors (e.g., GaN, GaInN, AlGaInP, AlGaN, GaAs, AlGaAs, AlGaP, GaP, GaAsP, GaInAs, InAs, InP, as well as combinations and alloys thereof) and II-VI semiconductors (e.g., ZnSe, CdSe, ZnCdSe, ZnTe, ZnTeSe, ZnS, ZnSSe, as well as combinations and alloys thereof). P-doped layer 310 may be doped with acceptor atoms that result in a relatively high hole concentration. Examples of suitable materials for p-doped layer 310 include, but are not limited to, magnesium-doped GaN. In some embodiments, p-doped layer 310 may comprise one or more layers, where each layer may have the same or different composition and the same or different thickness. N-doped layer 320 may be doped with donor atoms that result in a relatively high electron concentration. Examples of suitable materials for n-doped layer include, but are not limited to, silicon-doped GaN. In some embodiments, n-doped layer 320 may comprise one or more layers, where each layer may have the same or different composition and the same or different thickness. In some embodiments, p-doped layer 310 and n-doped layer 320 form a p-n junction where light-generating region 315 is disposed between p-doped layer 310 and n-doped layer 320.

In some embodiments, the light-generating region(s) of an LED (e.g., light-generating region 315) in a light-emitting region (e.g., a first light-emitting region, a second light-emitting region) comprise one or more light-generating materials. In some embodiments, the one or more light-generating materials comprise one or more semiconductor materials. Non-limiting examples of suitable semiconductor materials include III-V semiconductors (e.g., GaN, GaInN, AlGaInP, AlGaN, GaAs, AlGaAs, AlGaP, GaP, GaAsP, GaInAs, InAs, InP, as well as combinations and alloys thereof) and II-VI semiconductors (e.g., ZnSe, CdSe, ZnCdSe, ZnTe, ZnTeSe, ZnS, ZnSSe, as well as combinations and alloys thereof). In certain embodiments, the light-generating region comprises one or more quantum wells (e.g., arranged as layers) surrounded by barrier layers. The quantum well structure may be defined by a semiconductor material layer (e.g., in a single quantum well), or more than one semiconductor material layers (e.g., in multiple quantum wells), with a smaller electronic band gap as compared to the barrier layers. Non-limiting examples of suitable semiconductor material layers for the quantum well structures include InGaN, AlGaN, GaN, and combinations thereof (e.g., alternating InGaN/GaN layers, where a GaN layer serves as a barrier layer). In some embodiments, the light-generating region comprises other light-emitting materials, such as quantum dots and/or organic light-emitting materials (e.g., Alq₃).

The light-generating region of an LED in a light-emitting region (e.g., a first light-emitting region, a second light-emitting region) may generate light having any suitable peak wavelength. In certain preferred embodiments, the light-generating region generates light having a peak wavelength corresponding to blue light (e.g., having a peak wavelength of 430-480 nm). In certain other embodiments, the light-generating region generates light having a peak wavelength corresponding to cyan light (e.g., having a peak wavelength of 480-500 nm). The light-generating region may, in some embodiments, generate light having a peak wavelength corresponding to ultraviolet light (e.g., having a peak wavelength of 370-390 nm), violet light (e.g., having a peak wavelength of 390-430 nm), green light (e.g., having a peak wavelength of 500-550 nm), yellow-green (e.g., having a peak wavelength of 550-575 nm), yellow light (e.g., having a peak wavelength of 575-595 nm), amber light (e.g., having a peak wavelength of 595-605 nm), orange light (e.g., having a peak wavelength of 605-620 nm), red light (e.g., having a peak wavelength of 620-700 nm), and/or infrared light (e.g., having a peak wavelength of 700-1200 nm). In certain embodiments, the light-generating region of an LED is configured to emit light having a peak wavelength in a range from 390 nm to 430 nm, 400 nm to 430 nm, 400 nm and 500 nm, 430 nm and 480 nm, 440 nm to 460 nm, 440 nm and 480 nm, 440 nm and 460 nm, 480 nm and 500 nm, 500 nm to 550 nm, 550 nm to 575 nm, 575 nm to 595 nm, 595 nm to 605 nm, 600 nm to 700 nm, 605 nm to 620 nm, 610 nm to 630 nm, 620 nm to 650 nm, or 620 nm to 700 nm.

In some embodiments, an LED in a light-emitting region (e.g., a first light-emitting region, a second light-emitting region) of a light-emitting system is associated with one or more wavelength-converting materials configured to convert emitted light of a first wavelength (e.g., light generated by the light-generating region) to light of a second, different wavelength. In certain cases, the one or more wavelength-converting materials may be configured to convert non-white light into substantially white light. In some cases, the one or more wavelength-converting materials comprise at least 2, at least 3, at least 5, or at least 10 wavelength-converting materials.

In some instances, the one or more wavelength-converting materials comprise a downconverter (e.g., a material that converts emitted light of a shorter wavelength to light of a longer wavelength). In some instances, the one or more wavelength-converting materials comprise an upconverter (e.g., a material that converts emitted light of a longer wavelength to light of a shorter wavelength). The one or more wavelength-converting materials may comprise one or more phosphors, one or more quantum dots, and/or one or more ceramic materials. Each of the one or more wavelength-converting materials may be organic or inorganic. Each of the one or more wavelength-converting materials may have any suitable form (e.g., particles, platelets, a film).

The one or more wavelength-converting materials may be configured to absorb light having any suitable wavelength. In some embodiments, at least one (and, in some cases, each) wavelength-converting material is configured to absorb light having a peak wavelength in a range from 390 nm to 430 nm, 400 nm to 430 nm, 400 nm to 500 nm, 430 nm to 480 nm, 440 nm to 480 nm, 440 nm to 460 nm, 480 nm to 500 nm, 500 nm to 550 nm, 550 nm to 575 nm, 575 nm to 595 nm, 595 nm to 605 nm, 600 nm to 700 nm, 605 nm to 620 nm, 610 nm to 630 nm, 620 nm to 650 nm, or 620 nm to 700 nm.

The one or more wavelength-converting materials (e.g., phosphors) may be configured to emit light having any suitable wavelength. In some embodiments, at least one (and, in some cases, each) wavelength-converting material is configured to emit light having a peak wavelength corresponding to substantially green and/or yellow light. In certain instances, at least one (and, in some cases, each) wavelength-converting material is configured to emit light having a peak wavelength in a range from 390 nm to 430 nm, 400 nm to 430 nm, 400 nm to 500 nm, 430 nm to 480 nm, 440 nm to 480 nm, 440 nm to 460 nm, 480 nm to 500 nm, 500 nm to 520 nm, 500 nm to 550 nm, 500 nm to 600 nm, 540 nm to 560 nm, 550 nm to 575 nm, 575 nm to 595 nm, 595 nm to 605 nm, 600 nm to 700 mm, 605 nm to 620 nm, 610 nm to 630 nm, 620 nm to 650 nm, or 620 nm to 700 nm.

In some embodiments, at least one wavelength-converting material comprises a first phosphor. Any suitable phosphor may be used. Non-limiting examples of suitable phosphors include silicate phosphors, aluminate phosphors, nitride phosphors, oxynitride phosphors, phosphate phosphors, sulfide phosphors, and oxysulfide phosphors. In some embodiments, the first phosphor comprises a yellow phosphor. A non-limiting example of a suitable yellow phosphor is (Y, Gd, Lu, Sc, Sm, Tb, Th, Ir, Sb, Bi)₃(Al,Ga)₅O₁₂:Ce³⁺ (with or without Pr), sometimes referred to as a “YAG” (yttrium, aluminum, garnet) phosphor. In some embodiments, the yellow phosphor is a yellow silicate phosphor. A non-limiting example of a suitable yellow silicate phosphor is A[Srₓ(M₁)₁₋ₓ]_{z}SiO₄.(1-a)[Sr_{y}(M₂)_{1-y}]ᵤSiO₅:Eu²⁺D, where M₁ and M₂ are at least one of a divalent metal such as Ba, Mg, Ca, and Zn; 0.6≤a≤0.85; 0.3≤x≤0.6; 0.8≤y≤1; 1.5≤z≤2.5; 2.6≤u≤3.3:Eu and D are between 0.0001 and about 0.5; D is an anion selected form the group consisting of F, Cl, Br, S and N; and at least some of D replaces oxygen in the host lattice. In some embodiments, the yellow phosphor is a yellow nitride phosphor. Non-limiting examples of suitable yellow nitride phosphors include Ca-α-sialon:Eu²⁺ and CaAlSiN₃:Ce³⁺. Other types of yellow phosphors are also possible.

In some embodiments, the first phosphor comprises a red phosphor. The red phosphor may, according to some embodiments, comprise a red nitride phosphor (e.g., (Ca, Sr, Ba)AiSiN₃:Eu; (Ca,Sr,Ba)₂Si₅N₈:Eu; Sr[LiAl₃N₄]:Eu²⁺), a red fluoride phosphor (e.g., M₂XF₆:Mn⁴⁺ (M=Na, K; X=Si, Ge, Ti, Zr)), and/or a red sulfide phosphor (e.g., L₂O₂S:Eu³⁺, (Ca, Sr)S₁₋ₓSeₓ:Eu (0≤x≤1)⁺). Other types of red phosphors are also possible.

In some embodiments, the first phosphor comprises a green phosphor. Non-limiting examples of suitable green phosphors include ß-Sialon Si_{6-z}Al_{z}O_{z}N_{8-z} (0<z<4.2); SrSi₂O₂N₂:Eu; Sr₃Si₁₃Al₃O₂N₂₁; Ba₃Si₆O₁₂N₂:Eu²⁺; Lu₃Al₅O₁₂:Ce³⁺; Gd₃Al₅O₁₂:Ce³⁺; M₁₋ₓEuₓMg_{1-y}Mn_{y}Al_{z}O_{[(x+y)+3z/2)} where 0.1<x<1.0, 0.1<y<1.0, 0.2<x+y<2.0, and 2≤z≤14; (Sr,A₁)ₓ(Si,A₂)(O,A₃)₂₊ₓ:Eu²⁺ where A₁ is at least one divalent metal ion such as Mg, Ca, Ba, Zn or a combination of +1 and =3 ions, A₂ is a 3⁺, 4⁺ or 5⁺ cation including at least one of B, Al, Ga, C, Ge, P, A₃ is a 1⁻, 2⁻ or 3⁻ anion including F, Cl, and Br, and 1.5≤x≤2.5; (Ca, Sr, Ba)(Al, In, Ga)₂S₄:Eu²⁺; and ZnS:Cu,Al,Mn. Other green phosphors (e.g., green aluminate phosphors, green silicate phosphors, green sulfide phosphors) are also possible.

In some embodiments, the first phosphor comprises an orange phosphor.

Examples of suitable orange phosphors include, but are not limited to, orange oxynitride phosphors (e.g., Li₂SrSiON₂, which has an emission peak around 585 nm), yellow-orange nitride phosphors (e.g., CaAlSiN₃:Ce³⁺, which emits yellow-orange light around 580 nm); and orange-red aluminum-silicate phosphors (e.g., (Sr_{1-x-y}MₓT_{y})₃₋ₘEuₘ(Sii_{1-z}Al_{z})O₅ where M is at least one of Ba, Mg and Zn, T is a trivalent metal, 0≤x≤0.4, 0≤y≤0.4, 0≤z≤0.2 and 0.001≤m≤0.4). Other orange, yellow-orange, or orange-red phosphors are also possible.

In some embodiments, the first phosphor comprises a blue phosphor. Examples of suitable blue phosphors include, but are not limited to, aluminate-based blue phosphors (e.g., (M₁₋ₓEuₓ)_{2-z}Mg_{z}Al_{y})O_{[2+3/2)y} where M is at least one of Ba and Sr, (0.05<x<0.5; 3≤y≤8; and 0.8≤z≤1<1.2) or (0.2<x<0.5; 3≤y≤8; and 0.8≤z≤1<1.2) or (0.05<x<0.5; 3≤y≤12; and 0.8≤z≤1<1.2) or (0.2<x<0.5; 3≤y≤12; and 0.8≤z≤1<1.2) or (0.05<x<0.5; 3≤y≤6; and 0.8≤z≤1.2) and phosphate-based blue phosphors (e.g., (Sr,Ca,Ba,Mg)₁₀(PO₄)₆Cl:Eu²⁺). In some embodiments, the first phosphor comprises a blue-green phosphor (e.g., BaSi₂O₂N₂, which has an emission maximum around 494-504 nm with a full width half-maximum (FWHM) of 32 nm). Other blue or blue-green phosphors are also possible.

Other phosphor materials are also possible. Suitable phosphor materials have been described, for example, in U.S. Pat. No. 7,196,354, filed Sep. 29, 2005, entitled “Wavelength-converting Light-emitting Devices,” by Erchak, et al., which is incorporated herein by reference in its entirety.

In some embodiments, at least one of the wavelength-converting materials (e.g., phosphors) has particulate form. In some embodiments, the average particle size of the at least one wavelength-converting material (e.g., phosphor) is less than 100 micrometers (μm), less than 50 μm, less than 40 μm, less than 30 μm, less than 20 μm, less than 10 μm, less than 5 μm, less than 1 μm, less than 500 nm, less than 300 nm, less than 100 nm, less than 50 nm, less than 20 nm, or less than 10 nm. In some embodiments, the average particle size of the at least one wavelength-converting material is between 10 nm and 300 nm, between 300 nm and 1 μm, between 1 μm and 10 μm, between 4 μm and 16 μm, between 10 μm and 30 μm, or 30 μm and 100 μm. Other particle sizes are also possible.

In some embodiments, particles of at least one wavelength-converting material are distributed in a second material (e.g., an encapsulant or an adhesive, such as a silicone or an epoxy) to form a first wavelength-converting layer or a mixture for dispensing into an LED package.

In some embodiments, additional wavelength-converting materials (e.g., phosphors) may be added during post-processing packaging. For example, in the case of a device which requires one or more wavelength-converting materials, minor tuning with a single wavelength-converting material may be performed at the package level. In the case of a device which requires multiple wavelength-converting materials (e.g., a majority of yellow phosphor with a small quantity of a red phosphor to improve the CRI of the final device), one wavelength-converting material (e.g., a yellow phosphor) may be applied at the LED die level and another wavelength-converting material (e.g., a red phosphor) may be applied at the package level. Similarly, additional wavelength-converting materials may be added, in some embodiments, on top of the coating at the die level.

In some embodiments, an LED comprises electrically conductive layers. The electrically conductive layers (e.g., electrically conductive layers 305 and 325) may comprise any suitable electrically conductive material. Examples of suitable electrically conductive materials include, but are not limited to, silver, aluminum, copper, and gold.

In some embodiments, an LED comprises additional layers. As an illustrative example, a layer (e.g., an AlGaN layer) may be disposed between light-generating region 330 and p-doped layer 310. It should be understood that compositions other than those described herein may also be suitable for the layers of the LED.

Each light-emitting region of a light-emitting system may comprise one or more LEDs having a suitable combination of a light-generating material and a first wavelength-converting material and, in certain instances, a second wavelength-converting material. In some cases, LEDs of different light-emitting regions may have different light-generating materials, first wavelength-converting materials, and/or second wavelength-converting materials.

In some embodiments, a first light-generating material of one or more LEDs of a first light-emitting region is configured to emit light having a first peak wavelength and a second light-generating material of one or more LEDs of a second light-emitting region is configured to emit light having a second peak wavelength, and the absolute value of the difference between the first peak wavelength and the second peak wavelength is relatively small (e.g., 50 nm or less, 20 nm or less, 10 nm or less, 5 nm or less, 1 nm or less, or about 0 nm). In some embodiments, the absolute value of the difference between the first peak wavelength and the second peak wavelength is relatively large (e.g., at least 50 nm, at least 100 nm, at least 200 nm, at least 500 nm). In some cases, a light-emitting system comprises three or more light-emitting regions (e.g., at least 5 light-emitting regions, at least 10 light-emitting regions), wherein each light-emitting region comprises LEDs comprising light-generating materials, and the absolute value of the maximum difference between the peak wavelengths of the light-generating materials may be relatively small (e.g., 50 nm or less, 20 nm or less, 10 nm or less, 5 nm or less, 1 nm or less, or about 0 nm). In some embodiments, the absolute value of the maximum difference between the peak wavelengths of the light-generating materials may be relatively large (e.g., at least 50 nm, at least 100 nm, at least 200 nm, at least 500 nm).

In some embodiments, a first wavelength-converting material of one or more LEDs of a first light-emitting region is configured to absorb or emit light having a first peak wavelength and a first wavelength-converting material of one or more LEDs of a second light-emitting region is configured to absorb or emit light having a second peak wavelength, and the absolute value of the difference between the first peak wavelength and the second peak wavelength is relatively large (e.g., at least 50 nm, at least 100 nm, at least 200 nm, at least 500 nm). In some embodiments, the absolute value of the difference between the first peak wavelength and the second peak wavelength is relatively small (e.g., 50 nm or less, 20 nm or less, 10 nm or less, 5 nm or less, 1 nm or less, or about 0 nm). In some cases, a light-emitting system comprises three or more light-emitting regions (e.g., at least 5 light-emitting regions, at least 10 light-emitting regions), wherein each light-emitting region comprises LEDs associated with first wavelength-converting materials, and the absolute value of the minimum difference between the peak wavelengths absorbed or emitted by the first wavelength-converting materials may be relatively large (e.g., at least 50 nm, at least 100 nm, at least 200 nm, at least 500 nm). In certain embodiments, the absolute value of the maximum difference between the peak wavelengths absorbed or emitted by the first wavelength-converting materials may be relatively small (e.g., 50 nm or less, 20 nm or less, 10 nm or less, 5 nm or less, 1 nm or less, or about 0 nm).

In some embodiments, a second wavelength-converting material of one or more LEDs of a first light-emitting region is configured to absorb or emit light having a first peak wavelength and a second wavelength-converting material of one or more LEDs of a second light-emitting region is configured to absorb or emit light having a second peak wavelength, and the absolute value of the difference between the first peak wavelength and the second peak wavelength is relatively large (e.g., at least 50 nm, at least 100 nm, at least 200 nm, at least 500 nm). In some embodiments, the absolute value of the difference between the first peak wavelength and the second peak wavelength is relatively small (e.g., 50 nm or less, 20 nm or less, 10 nm or less, 5 nm or less, 1 nm or less, or about 0 nm). In some cases, a light-emitting system comprises three or more light-emitting regions (e.g., at least 5 light-emitting regions, at least 10 light-emitting regions), wherein each light-emitting region comprises LEDs associated with second wavelength-converting materials, and the absolute value of the minimum difference between the peak wavelengths absorbed or emitted by the second wavelength-converting materials may be relatively large (e.g., at least 50 nm, at least 100 nm, at least 200 nm, at least 500 nm). In certain embodiments, the absolute value of the maximum difference between the peak wavelengths absorbed or emitted by the second wavelength-converting materials may be relatively small (e.g., 50 nm or less, 20 nm or less, 10 nm or less, 5 nm or less, 1 nm or less, or about 0 nm).

In some cases, a combination of a light-generating material and one or more wavelength-converting materials may emit light having a relatively low melanopic ratio. As an illustrative, non-limiting example, an LED configured to emit light having a relatively low melanopic ratio may comprise a light-generating material configured to emit blue light, a first wavelength-converting material configured to emit yellow light, and a second wavelength-converting material configured to emit red light. In particular, the LED may comprise a light-generating material configured to emit light having a peak wavelength around 450 nm, a first wavelength material configured to emit light having a peak wavelength around 550 nm, and a second wavelength material configured to emit light having a peak wavelength around 630 nm.

In some cases, a combination of a light-generating material and one or more wavelength-converting materials may emit light having a relatively high melanopic ratio. As one illustrative, non-limiting example, an LED configured to emit light having a relatively high melanopic ratio may comprise a light-generating material configured to emit blue light, a first wavelength-converting material configured to emit green light, and a second wavelength-converting material configured to emit red light. In particular, the LED may comprise a light-generating material configured to emit light having a peak wavelength around 450 nm, a first wavelength material configured to emit light having a peak wavelength around 510 nm, and a second wavelength material configured to emit light having a peak wavelength around 620 nm. As another illustrative, non-limiting example, an LED configured to emit light having a relatively high melanopic ratio may comprise a light-generating material configured to emit cyan light, a first wavelength-converting material configured to emit green light, and a second wavelength-converting material configured to emit red light. In particular, the LED may comprise a light-generating material configured to emit light having a peak wavelength around 490 nm, a first wavelength material configured to emit light having a peak wavelength around 510 nm, and a second wavelength material configured to emit light having a peak wavelength around 620 nm. In certain embodiments, a light-emitting region may comprise both LEDs comprising a first light-generating material configured to emit blue light (e.g., light having a peak wavelength around 450 nm) and LEDs comprising a second light-generating material configured to emit cyan light (e.g., light having a peak wavelength around 490 nm).

In some embodiments, each light-emitting region of a light-emitting system may have an electrical contact formed on a surface of each region. The electrical contacts function to provide power to the light-emitting regions to generate light. Such a contact arrangement may enable current to be provided independently to the light-emitting regions. That is, the current to a first light-emitting region may be provided independently from the current provided to a second light-emitting region. Thus, the light-emitting regions are referred to as being independently electrically addressable. In these embodiments, different current levels may be provided to the different light-emitting regions. This may enable light having different circadian and/or color characteristics to be emitted from each region.

Any suitable package configuration may be employed in embodiments described herein. Non-limiting examples of suitable packages include lead frame or ceramic packages (e.g., lead or ceramic frames comprising one or more cavities), surface-mounted packages, and chip-on-board packages. In some embodiments, the system comprises a chip-on-board package. A chip-on-board package generally refers to a system in which a plurality of LED chips (as opposed to packaged LEDs) are bonded to a substrate. In some cases, the largest nearest-neighbor distance between LED chips in a chip-on-board package may be less than the largest nearest-neighbor distance between individually packaged LEDs in a comparable surface-mounted package. In some cases, having smaller nearest neighbor distances may advantageously increase the output and/or efficiency of a light-emitting system.

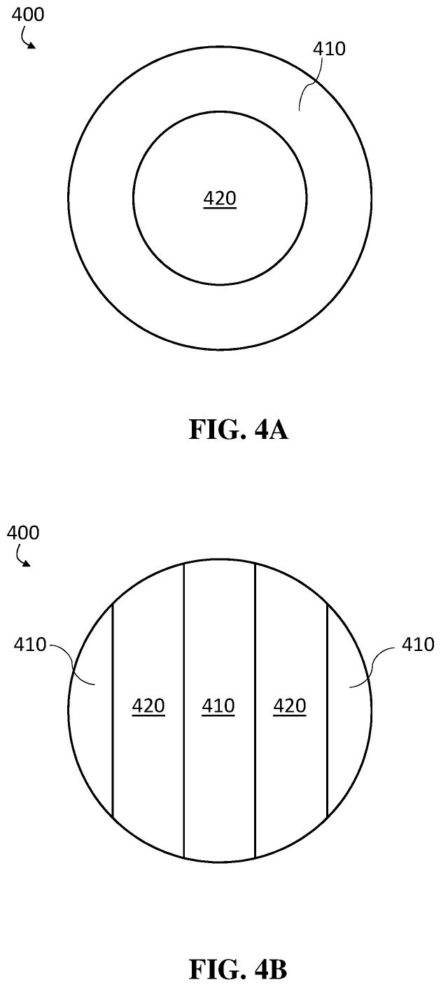

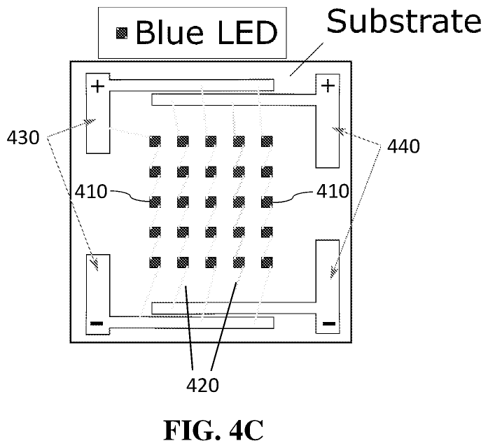

In some embodiments, a chip-on-board package comprises two or more regions that are separated from each other (e.g., by confinement materials). The regions may have any suitable shape or configuration. As a non-limiting, illustrative example, FIG. 4A shows an exemplary chip-on-board package 400 comprising first circular region 410 and second circular region 420. As another non-limiting, illustrative example, FIG. 4B shows an exemplary chip-on-board package 400 comprising first striped regions 410 and second striped regions 420. In some cases, first striped regions 410 may be in electrical communication with a first set of electrodes, and second striped regions 420 may be in electrical communication with a second set of electrodes. For example, as shown in FIG. 4C, first striped regions 410 may be in electrical communication (e.g., via wire bonds) with first electrodes 430, and second striped regions 420 may be electrical communication (e.g., via wire bonds) with second electrodes 440.

In some embodiments, light emitted from first region(s) 410 (e.g., a first circular region, first striped regions) may have a first melanopic ratio, and light emitted from second region(s) 420 (e.g., a second circular region, second striped regions) may have a second melanopic ratio that is different from the first melanopic ratio. In some embodiments, light emitted from first region(s) 410 may have substantially similar CCT and/or CRI values as light emitted from second region(s) 420.

In some embodiments, first region(s) 410 may comprise a first plurality of LED chips. In some instances, one or more LED chips of the first plurality of LED chips comprise at least one light-generating region comprising a first light-generating material. In certain cases, one or more LED chips of the first plurality of LED chips are associated with at least one wavelength-converting layer comprising a first wavelength-converting material. For example, the at least one wavelength-converting layer may be coated or otherwise deposited on the one or more LED chips of the first plurality of LED chips. In some embodiments, second region(s) 420 may comprise a second plurality of LED chips. In some instances, one or more LED chips of the second plurality of LED chips comprise at least one light-generating region comprising a second light-generating material. In certain cases, one or more LED chips of the second plurality of LED chips are associated with at least one wavelength-converting layer comprising a second wavelength-converting material. For example, the at least one wavelength-converting layer may be coated or otherwise deposited on the second plurality of LED chips. The first light-generating material of the first plurality of LED chips and the second light-generating material of the second plurality of LED chips may be the same or different. Similarly, the first wavelength-converting material of the first plurality of LED chips and the second wavelength-converting material of the second plurality of LED chips may be the same or different. In some embodiments, the first plurality of LED chips and/or the second plurality of LED chips may each comprise a plurality of wavelength-converting layers. In some such embodiments, the wavelength-converting material(s) in the plurality of wavelength-converting layers of the first plurality of the LED chips and the wavelength-converting material(s) in the plurality of wavelength-converting layers of the second plurality of LED chips may be the same or different.

In operation, current may be directed to flow to the first plurality of LEDs in first region(s) 410 during a first time period. In some instances, light having a first melanopic ratio, a first CCT value, and a first CRI value may be emitted during the first time period. During a second time period, current may be directed to flow to the second plurality of LEDs in second region(s) 420. In some instances, light having a second melanopic ratio, a second CCT value, and a second CRI value may be emitted during the second time period. In some embodiments, the second melanopic ratio is different from the first melanopic ratio. In certain embodiments, the absolute value of the difference between the first melanopic ratio and the second melanopic ratio is relatively large (e.g., at least 0.1). In some cases, the second CCT value is substantially similar to the first CCT value. In certain embodiments, the absolute value of the difference between the first CCT value and the second CCT value is relatively small (e.g., 1000 K or less). In some cases, the second CRI value is substantially similar to the first CRI value. In certain embodiments, the absolute value of the difference between the first CRI value and the second CRI value is relatively small (e.g., less than 10). In some instances, current may be selectively directed to flow to LEDs configured to emit light having a desired melanopic ratio.

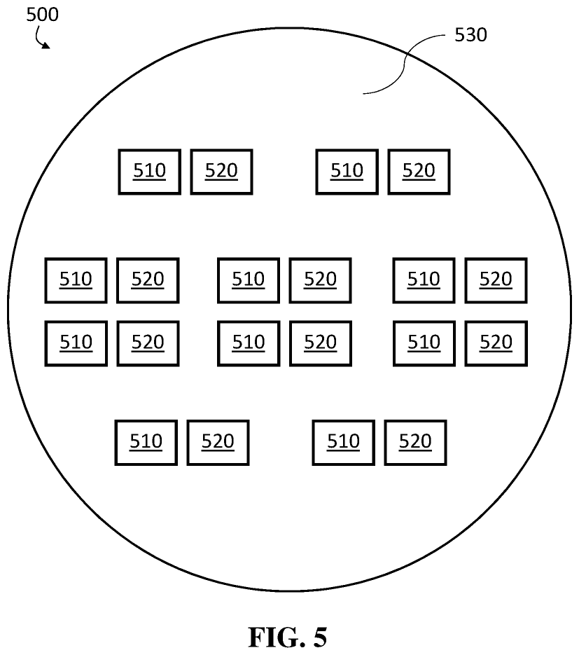

In some embodiments, a light-emitting system comprises a surface-mounted LED package. FIG. 5 illustrates an exemplary system comprising a surface-mounted LED package. As shown in FIG. 5, system 500 comprises printed circuit board (PCB) 530. In some embodiments, a plurality of first packaged LEDs 510 may be bonded to PCB 530. In some instances, one or more, or in some cases all, LEDs of the plurality of first packaged LEDs 510 are configured to emit light having a first melanopic ratio, a first CCT value, and/or a first CRI value. In some embodiments, a plurality of second packaged LEDs 520 may be bonded to PCB 530. In some instances, one or more, or in some cases all, LEDs of the plurality of second packaged LEDs 510 are configured to emit light having a second melanopic ratio, a second CCT value, and/or a second CRI value. In some embodiments, the second melanopic ratio is different from the first melanopic ratio. In certain embodiments, the absolute value of the difference between the first melanopic ratio and the second melanopic ratio is relatively large (e.g., at least 0.1). In some cases, the second CCT value is substantially similar to the first CCT value. In certain embodiments, the absolute value of the difference between the first CCT value and the second CCT value is relatively small (e.g., 1000 K or less). In some cases, the second CRI value is substantially similar to the first CRI value. In certain embodiments, the absolute value of the difference between the first CRI value and the second CRI value is relatively small (e.g., less than 10). In some instances, current may be selectively directed to flow to packaged LEDs configured to emit light having a desired melanopic ratio.

First packaged LEDs 510 and second packaged LEDs 520 may be arranged in any suitable configuration. In certain instances, copper traces may be deposited on PCB 530 to obtain an electrical circuit. In some embodiments, circadian effect tunability may be achieved by adjusting the current directed to first packaged LEDs 510 and/or second packaged LEDs 520.

Certain aspects are directed to a method of emitting light. In some embodiments, the method comprises emitting light having a first melanopic ratio, a first CCT value, and/or a first CRI value. In some such embodiments, this step may be performed by directing current to flow to a first light-emitting region comprising one or more LEDs configured to emit light having a first melanopic ratio, a first CCT value, and/or a first CRI value. In some embodiments, the method comprises emitting light having a second melanopic ratio, a second CCT value, and/or a second CRI value. In some such embodiments, this step may be performed by directing current to flow to a second light-emitting region comprising one or more LEDs configured to emit light having a second melanopic ratio, a second CCT value, and/or a second CRI value. In some embodiments, the second melanopic ratio is different from the first melanopic ratio. In certain embodiments, the absolute value of the difference between the first melanopic ratio and the second melanopic ratio is relatively large (e.g., at least 0.1). In some cases, the second CCT value is substantially similar to the first CCT value. In certain embodiments, the absolute value of the difference between the first CCT value and the second CCT value is relatively small (e.g., 1000 K or less). In some cases, the second CRI value is substantially similar to the first CRI value. In certain embodiments, the absolute value of the difference between the first CRI value and the second CRI value is relatively small (e.g., less than 10).

The systems and methods described herein can be used in a variety of lighting applications. In some embodiments, for example, devices described herein may be used for illumination (e.g., illumination of at least a portion of a room) or for electronic displays (e.g., cell phone displays, computer monitors, display projectors).

While several embodiments of the present invention have been described and illustrated herein, those of ordinary skill in the art will readily envision a variety of other means and/or structures for performing the functions and/or obtaining the results and/or one or more of the advantages described herein, and each of such variations and/or modifications is deemed to be within the scope of the present invention. More generally, those skilled in the art will readily appreciate that all parameters, dimensions, materials, and configurations described herein are meant to be exemplary and that the actual parameters, dimensions, materials, and/or configurations will depend upon the specific application or applications for which the teachings of the present invention is/are used. Those skilled in the art will recognize, or be able to ascertain using no more than routine experimentation, many equivalents to the specific embodiments of the invention described herein. It is, therefore, to be understood that the foregoing embodiments are presented by way of example only and that, within the scope of the appended claims and equivalents thereto, the invention may be practiced otherwise than as specifically described and claimed. The present invention is directed to each individual feature, system, article, material, kit, and/or method described herein. In addition, any combination of two or more such features, systems, articles, materials, kits, and/or methods, if such features, systems, articles, materials, kits, and/or methods are not mutually inconsistent, is included within the scope of the present invention.

Having thus described several aspects of at least one embodiment, it is to be appreciated that various alterations, modifications, and improvements will readily occur to those skilled in the art. Such alterations, modifications, and improvements are intended to be within the spirit and scope of the present disclosure. Accordingly, the foregoing description and drawings are by way of example only.

The above-described embodiments of the present disclosure can be implemented in any of numerous ways. For example, the embodiments may be implemented using hardware, software or a combination thereof. When implemented in software, the software code can be executed on any suitable processor or collection of processors, whether provided in a single computer or distributed among multiple computers.

Also, the various methods or processes outlined herein may be coded as software that is executable on one or more processors that employ any one of a variety of operating systems or platforms. Additionally, such software may be written using any of a number of suitable programming languages and/or programming or scripting tools, and also may be compiled as executable machine language code or intermediate code that is executed on a framework or virtual machine.

In this respect, the concepts disclosed herein may be embodied as a non-transitory computer-readable medium (or multiple computer-readable media) (e.g., a computer memory, one or more floppy discs, compact discs, optical discs, magnetic tapes, flash memories, circuit configurations in Field Programmable Gate Arrays or other semiconductor devices, or other non-transitory, tangible computer storage medium) encoded with one or more programs that, when executed on one or more computers or other processors, perform methods that implement the various embodiments of the present disclosure discussed above. The computer-readable medium or media can be transportable, such that the program or programs stored thereon can be loaded onto one or more different computers or other processors to implement various aspects of the present disclosure as discussed above.

The terms “program” or “software” are used herein to refer to any type of computer code or set of computer-executable instructions that can be employed to program a computer or other processor to implement various aspects of the present disclosure as discussed above. Additionally, it should be appreciated that according to one aspect of this embodiment, one or more computer programs that when executed perform methods of the present disclosure need not reside on a single computer or processor, but may be distributed in a modular fashion amongst a number of different computers or processors to implement various aspects of the present disclosure.

Computer-executable instructions may be in many forms, such as program modules, executed by one or more computers or other devices. Generally, program modules include routines, programs, objects, components, data structures, etc. that perform particular tasks or implement particular abstract data types. Typically the functionality of the program modules may be combined or distributed as desired in various embodiments.

Also, data structures may be stored in computer-readable media in any suitable form. For simplicity of illustration, data structures may be shown to have fields that are related through location in the data structure. Such relationships may likewise be achieved by assigning storage for the fields with locations in a computer-readable medium that conveys relationship between the fields. However, any suitable mechanism may be used to establish a relationship between information in fields of a data structure, including through the use of pointers, tags or other mechanisms that establish relationship between data elements.

Various features and aspects of the present disclosure may be used alone, in any combination of two or more, or in a variety of arrangements not specifically discussed in the embodiments described in the foregoing and is therefore not limited in its application to the details and arrangement of components set forth in the foregoing description or illustrated in the drawings. For example, aspects described in one embodiment may be combined in any manner with aspects described in other embodiments.

Also, the concepts disclosed herein may be embodied as a method, of which an example has been provided. The acts performed as part of the method may be ordered in any suitable way. Accordingly, embodiments may be constructed in which acts are performed in an order different than illustrated, which may include performing some acts simultaneously, even though shown as sequential acts in illustrative embodiments.

Use of ordinal terms such as “first,” “second,” “third,” etc. in the claims to modify a claim element does not by itself connote any priority, precedence, or order of one claim element over another or the temporal order in which acts of a method are performed, but are used merely as labels to distinguish one claim element having a certain name from another element having a same name (but for use of the ordinal term) to distinguish the claim elements.

Also, the phraseology and terminology used herein is for the purpose of description and should not be regarded as limiting. The use of “including,” “comprising,” “having,” “containing,” “involving,” and variations thereof herein, is meant to encompass the items listed thereafter and equivalents thereof as well as additional items.

All definitions, as defined and used herein, should be understood to control over dictionary definitions, definitions in documents incorporated by reference, and/or ordinary meanings of the defined terms.

The indefinite articles “a” and “an,” as used herein in the specification and in the claims, unless clearly indicated to the contrary, should be understood to mean “at least one.”

The phrase “and/or,” as used herein in the specification and in the claims, should be understood to mean “either or both” of the elements so conjoined, i.e., elements that are conjunctively present in some cases and disjunctively present in other cases. Multiple elements listed with “and/or” should be construed in the same fashion, i.e., “one or more” of the elements so conjoined. Other elements may optionally be present other than the elements specifically identified by the “and/or” clause, whether related or unrelated to those elements specifically identified. Thus, as a non-limiting example, a reference to “A and/or B”, when used in conjunction with open-ended language such as “comprising” can refer, in one embodiment, to A only (optionally including elements other than B); in another embodiment, to B only (optionally including elements other than A); in yet another embodiment, to both A and B (optionally including other elements); etc.

As used herein in the specification and in the claims, “or” should be understood to have the same meaning as “and/or” as defined above. For example, when separating items in a list, “or” or “and/or” shall be interpreted as being inclusive, i.e., the inclusion of at least one, but also including more than one, of a number or list of elements, and, optionally, additional unlisted items. Only terms clearly indicated to the contrary, such as “only one of” or “exactly one of,” or, when used in the claims, “consisting of,” will refer to the inclusion of exactly one element of a number or list of elements. In general, the term “or” as used herein shall be interpreted as having the same meaning as “and/or” as defined above and shall not be interpreted as indicating exclusive alternatives (i.e. “one or the other but not both”) unless preceded by terms of exclusivity, such as “either,” “one of,” “only one of,” or “exactly one of.” “Consisting essentially of,” when used in the claims, shall have its ordinary meaning as used in the field of patent law.

As used herein in the specification and in the claims, the phrase “at least one,” in reference to a list of one or more elements, should be understood to mean at least one element selected from any one or more of the elements in the list of elements, but not necessarily including at least one of each and every element specifically listed within the list of elements and not excluding any combinations of elements in the list of elements. This definition also allows that elements may optionally be present other than the elements specifically identified within the list of elements to which the phrase “at least one” refers, whether related or unrelated to those elements specifically identified. Thus, as a non-limiting example, “at least one of A and B” (or, equivalently, “at least one of A or B,” or, equivalently “at least one of A and/or B”) can refer, in one embodiment, to at least one, optionally including more than one, A, with no B present (and optionally including elements other than B); in another embodiment, to at least one, optionally including more than one, B, with no A present (and optionally including elements other than A); in yet another embodiment, to at least one, optionally including more than one, A, and at least one, optionally including more than one, B (and optionally including other elements); etc.

As used herein, when a structure (e.g., layer, region) is referred to as being “on”, “over” “overlying” or “supported by” another structure, it can be directly on the structure, or an intervening structure (e.g., layer, region) also may be present. A structure that is “directly on” or “in contact with” another structure means that no intervening structure is present.

It should also be understood that, unless clearly indicated to the contrary, in any methods claimed herein that include more than one step or act, the order of the steps or acts of the method is not necessarily limited to the order in which the steps or acts of the method are recited.

In the claims, as well as in the specification above, all transitional phrases such as “comprising,” “including,” “carrying,” “having,” “containing,” “involving,” “holding,” “composed of,” and the like are to be understood to be open-ended, i.e., to mean including but not limited to. Only the transitional phrases “consisting of” and “consisting essentially of” shall be closed or semi-closed transitional phrases, respectively, as set forth in the United States Patent Office Manual of Patent Examining Procedures, Section 2111.03.

What is claimed is:

1. A light-emitting system, comprising:
a first light-emitting region configured to emit light having a first melanopic ratio and a first correlated color temperature (CCT) value; and
a second light-emitting region configured to emit light having a second melanopic ratio and a second CCT value,
wherein the first melanopic ratio and the second melanopic ratio have a difference of at least 0.1, and
wherein the first CCT value and the second CCT value have a difference of 1000 K or less.

2. The light-emitting system of claim 1, wherein the first melanopic ratio and the second melanopic ratio have a difference in a range from 0.1 to 1.0.

3. The light-emitting system of claim 1, wherein the first melanopic ratio and the second melanopic ratio have a difference of at least 0.3.

4-7. (canceled)

8. The light-emitting system of claim 1, wherein the first CCT value and the second CCT value have a difference of 500 K or less.

9. The light-emitting system of claim 1, wherein the first CCT value and the second CCT value are in a range from 1000 K to 3000 K.

10. The light-emitting system of claim 1, wherein the first CCT value and the second CCT value are in a range from 3500 K to 6500 K.

11. The light-emitting system of claim 1, wherein the first light-emitting region has a first color rendering index (CRI) value and the second light-emitting region has a second CRI value, wherein the first CRI value and the second CRI value are at least 70.

12-16. (canceled)

17. The light-emitting system of claim 1, wherein the first light-emitting region comprises a first light-emitting diode (LED) comprising a light-generating region configured to emit light having a peak wavelength in a range from 380 nm to 480 nm.

18. The light-emitting system of claim 1, wherein the first light-emitting region comprises a first light-emitting diode (LED), and wherein the first LED of the first light-emitting region is associated with a first wavelength-converting material configured to emit light having a first peak wavelength.

19. The light-emitting system of claim 17, wherein the first LED of the first light-emitting region is associated with a first wavelength-converting material configured to emit light having a peak wavelength in a range from 500 nm to 600 nm and/or a second wavelength-converting material configured to emit light having a peak wavelength in a range from 600 nm to 700 nm.

20. The light-emitting system of claim 19, wherein the first wavelength-converting material is configured to emit light having a peak wavelength in a range from 500 nm to 560 nm and the second wavelength-converting material is configured to emit light having a peak wavelength in a range from 620 nm to 700 nm.

21. The light-emitting system of claim 18, wherein the first LED of the first light-emitting region is associated with a second wavelength-converting material configured to emit light having a second peak wavelength, where the second peak wavelength is different from the first peak wavelength.

22-26. (canceled)

27. The light-emitting system of claim 1, wherein the second light-emitting region comprises a first light-emitting diode (LED) comprising a light-generating region configured to emit light having a peak wavelength in a range from 380 nm to 480 nm.

28. (canceled)

29. The light-emitting system of claim 1, wherein the second light-emitting region comprises a first light-emitting diode (LED), wherein the first LED of the second light-emitting region is associated with a first wavelength-converting material configured to emit light having a first wavelength and/or a second wavelength-converting material configured to emit light having a second wavelength different from the first wavelength.

30. The light-emitting system of claim 27, wherein the first LED of the second light-emitting region is associated with a first wavelength-converting material configured to emit light having a peak wavelength in a range from 500 nm to 600 nm and/or a second wavelength-converting material configured to emit light having a peak wavelength in a range from 600 nm and 700 nm.

31-36. (canceled)

37. The light-emitting system of claim 27, wherein the second light-emitting region further comprises a second LED comprising a light-emitting region configured to emit light having a peak wavelength in a range from 480 nm to 500 nm.

38. The light-emitting system of claim 1, wherein the first light-emitting region and the second light-emitting region are configured to emit substantially white light.

39. The light-emitting system of claim 1, wherein the first light-emitting region comprises a first plurality of LEDs and/or the second light-emitting region comprises a second plurality of LEDs.

40. A light-emitting system, comprising:
a first light-emitting region comprising a first LED associated with a first wavelength-converting material; and
a second light-emitting region comprising a second LED associated with a second wavelength-converting material,
wherein a first combination comprising the first LED and the first wavelength-converting material is configured to emit light having a first melanopic ratio and a first correlated color temperature (CCT) value,
wherein a second combination comprising the second LED and the second wavelength-converting material is configured to emit light having a second melanopic ratio and a second CCT value,
wherein the first melanopic ratio and the second melanopic ratio have a difference of at least 0.1, and
wherein the first CCT value and the second CCT value have a difference of 1000 K or less.

41. A method, comprising:
emitting light having a first melanopic ratio and a first CCT value; and
emitting light having a second melanopic ratio and a second CCT value,
wherein the first melanopic ratio and the second melanopic ratio have a difference of at least 0.1, and
wherein the first CCT value and the second CCT value have a difference of 1000 K or less.
